(19) United States
(12) Patent Application Publication
(10) Pub. No.: US 2018/0122509 A1
(43) Pub. Date: May 3, 2018

(54) Multilevel Intelligent Interactive Mobile Health System for Behavioral Physiology Self-Regulation in Real-Time

(71) Applicant: Talislife Sàrl, Nyon (CH)

(72) Inventor: Chris Christiansson, Gengins (CH)

(21) Appl. No.: 15/798,631

(22) Filed: Oct. 31, 2017

Related U.S. Application Data
(60) Provisional application No. 62/414,812, filed on Oct. 31, 2016.

(51) Int. Cl. G16H 50/20; G16H 10/20; G16H 50/30; G16H 40/67

(52) U.S. Cl. CPC G16H 50/20; G16H 40/67; G16H 50/30; G16H 10/20

(57) ABSTRACT

An automated method for personal health management of a user having a personal user profile, the method including repeatedly measuring a plurality of health parameters by a mobile electronic device, the health parameters including a heart rate variability, a blood pressure, a motion activity, and a weight of the user, calculating a base line dataset by the computing device for each one of the plurality of health parameters for a predetermined period of time, comparing recently measured health parameters from the step of repeatedly measuring with the base line dataset, providing on a display that is operatively connected to the computing device a first raw feedback on a health performance of the user based on the step of comparing, repeatedly prompting the user to answer contextual questions, the contextual questions related to at least one of physical, mental, emotional, and behavioral status of the user, and generating a second detailed feedback based on the personal user profile, the baseline data set, the measured health parameters, and a given context, to determine values for a plurality of health segments, and providing the second detailed feedback to the display of the user, based on a request by the user that includes the given context and data of the plurality of health segments.

### CROSS-REFERENCE TO RELATED APPLICATIONS

The present invention claims priority to the U.S. provisional patent application with the Ser. No. 62/414,812 that was filed on Oct. 31, 2016, the entire contents thereof being herewith incorporated by reference.

### FIELD OF THE INVENTION

The present invention is related to the field of physical, mental, and emotional health status and health progress measurement, and systems, devices, and methods related to the same.

### BACKGROUND

A big number of medical care focuses is on the illness stage. To a large extent, the disease that are treated almost exclusively are in the chronic illness stage or in the later stages. Today clinicians and health professionals lack sensitive tools and systems to determine individual, and also multilevel baselines of health parameters and early detection of a potential chronic illness. Individuals frequently deny the need for checking their health parameters and act on early signs of a possible unhealthy behavior or illness as they lack tools for self-monitoring with personalized system generating feedback and interpretations of early detection of a problem, recovery solutions, better prevention and monitoring of vital health parameters.

It is a trend of shifting more attention and resources to preventive care. Early detection of issues before a problem can save many peoples pain from chronic illnesses and life's and even more important it will cure people early and apply additional solutions as part of a preventive health care process. It is also a trend to put more self-responsibility on the human.

Quantified-self trends, usually are not accurate enough and no built in feedback and action system. Biofeedback is good measure but needs a skilled therapist to properly evaluate. Therefore, in light of all the drawbacks of exiting solutions for health assessment and health progress management, better and novel solutions are strongly desired.

### SUMMARY

According to one aspect of the present invention, an automated method, system, or software recorded on a computer-readable medium is provided, the method for personal health management of a user having a personal user profile, the method performed on a computing device.

Preferably, the method includes the steps of repeatedly measuring a plurality of health parameters by a mobile electronic device, the health parameters including a heart rate variability, a blood pressure, a motion activity, and a weight of the user, calculating a base line dataset by the computing device for each one of the plurality of health parameters for a predetermined period of time, comparing recently measured health parameters from the step of repeatedly measuring with the base line dataset, and providing on a display that is operatively connected to the computing device a first raw feedback on a health performance of the user based on the step of comparing.

Moreover, the method further preferably includes the steps of repeatedly prompting the user to answer contextual questions, the contextual questions related to at least one of physical, mental, emotional, and behavioral status of the user, and generating a second detailed feedback based on the personal user profile, the baseline data set, the measured health parameters, and a given context, to determine values for a plurality of health segments, and providing the second detailed feedback to the display of the user, based on a request by the user that includes the given context and data of the plurality of health segments.

According to another aspect of the present invention, accuracy in measuring, personalized feedback and action system skilled algorithm to interpret and provide training solutions is provided. According to some aspects, the features of the present invention can focus on different health segments, for example ten (10) health segments by an integrated in an intelligent multilevel mobile health system for behavioral physiology self-regulation involving a vast set of sensors already existing in a smartphone or linked to external devices, and intelligent application that process data and provide from accessing data stored on the phone or the cloud with personalized feedback, solutions, training protocols and monitoring of progress and early detection.

By monitoring different heath parameters that are related to different health segments representing human health risk and behaviors, and provide personalized interpretations and feedback on what to do and how with a monitoring process to re-insure good recovery with major savings, pain can be gained, and prevention through self-learning for happen again. The health segments can be chosen to represent a large percentage of health issues and consequences, for example ten (10) health segments are responsible for twenty (20) of the most known chronic illness which account for 80% of the total health care costs.

According to another aspect of the present invention, the system interaction between the smartphone of the users, measuring sensors in the smartphone and external health devices, cloud servers and the user him/her self develops related protocols to train them to become more aware and apply validated personalized trained self-regulation protocols which over time when repeated training have occurred successfully high in self-efficacy become trained automated response patterns (TARP).

The above and other objects, features and advantages of the present invention and the manner of realizing them will become more apparent, and the invention itself will best be understood from a study of the following description with reference to the attached drawings showing some preferred embodiments of the invention.

### BRIEF DESCRIPTION OF THE SEVERAL VIEWS OF THE DRAWINGS

The accompanying drawings, which are incorporated herein and constitute part of this specification, illustrate the presently preferred embodiments of the invention, and together with the general description given above and the detailed description given below, serve to explain features of the invention.

FIGS. 1A and 1B schematically show a system 10 with different devices connected thereto, and different software elements that can be performed by the respective devices, according to an aspect of the present invention, and FIG. 1C shows an exemplary chart of the ten (10) health segments that influence the human health risks and behavior that can be analyzed by system 10 and method 20;

FIG. 2 schematically shows method 20 for being performed with system 10, with different method steps, according to an aspect of the present invention;

FIG. 3 schematically shows a different methods steps of the algorithm for analyzing the health parameters of step S70, the answers to prompted questions, and the user profile, according to another aspect of the present invention;

FIG. 4A to 4C show graphical representations of the method steps performed in step S70, showing a decision tree DT in FIG. 4A, a knowledge graph KG in FIG. 4B, and a neural network NN shown in FIG. 4C, according to another aspect of the present invention.

FIG. 5 schematically shows a system 10 having a smartphone 100, and different method steps and their respective order of method 20 when performed on system 10;

FIG. 6 shows different human health risks and behaviors;

FIG. 7 schematically shows a flowchart representing a method of trained instant action solutions (TIAS) and trained automated response patterns (TARP) behavioral physiological self-regulation (BPSR) according to another aspect of the present invention;

FIG. 8A to 8C exemplarily show steps of the method for initializing the system 10 and method 20 by a first-time self-regulation (FT-SR) method for a first-time user, including the user touching a camera 107 of smartphone 100 (FIG. 8A), and a first part of the steps of the FT-SR method (FIG. 8B), and a second part of FT-SR method (FIG. 8C);

FIG. 9 schematically shows an early self-regulation (SR) chart;

FIGS. 10 and 11 schematically depict charts for system recognize behavioral physiological parameters (SRBPP) for the method;

FIG. 12 schematically depicts the self-therapy journey (STJ) chart for the method;

FIG. 13 schematically depicts another chart for System Recognize Behavioral Physiological Parameters (SRBPP) for the method;

FIG. 14 schematically depicts a standard instant action solution process according to an aspect of the present invention;

FIG. 15 shows an exemplary contextual grid for classifying different events; and

FIG. 16 shows an exemplary human performance curve that can serve as a benchmark for the present system 10 and method 20.

Herein, identical reference numerals are used, where possible, to designate identical elements that are common to the figures. Also, the images are simplified for illustration purposes and may not be depicted to scale.

### DETAILED DESCRIPTION OF THE SEVERAL EMBODIMENTS

According to one aspect of the present invention, a system 10 is provided, including an electronic device, for example a smartphone 100 having a software application performed thereon, a network-connected cloud server 103 having a database 130 operatively connected thereto, and the performance of self-regulation methods for real-time training based on one or multiple measured behavioral-physiology parameters, for example health parameters HP. Health parameters can be measured via sensors 106, 107, and a sophisticated intelligent software, application program interface, activity-event-feeling determination questions provided to the user and contextual inputs can be provided. Based on these data values, it is possible to calculate weights and linking weights for and between different health segments. Also, a non-transitory computer readable medium can be provided, having computer instructions recorded thereon, the computer instructions configured to perform at least one or more steps of the method 20 as further described herein.

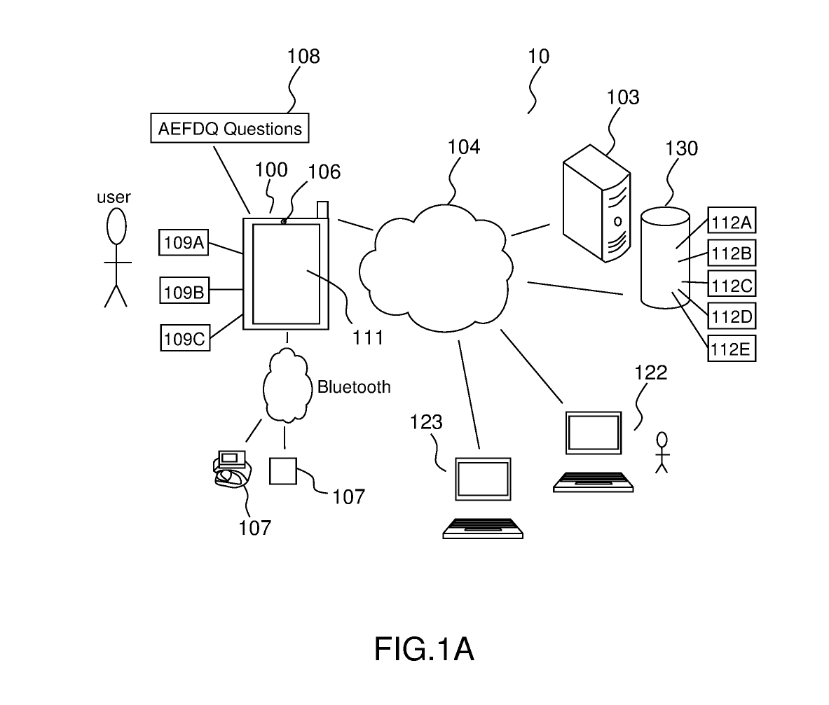

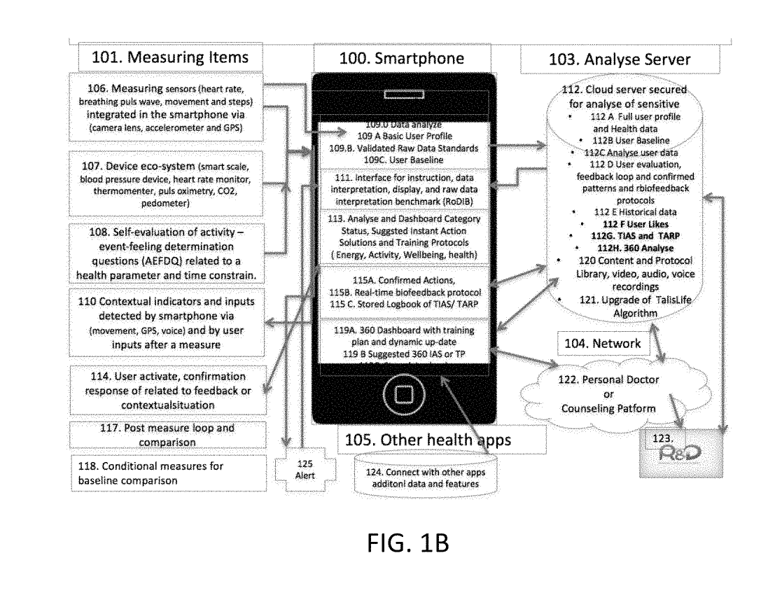

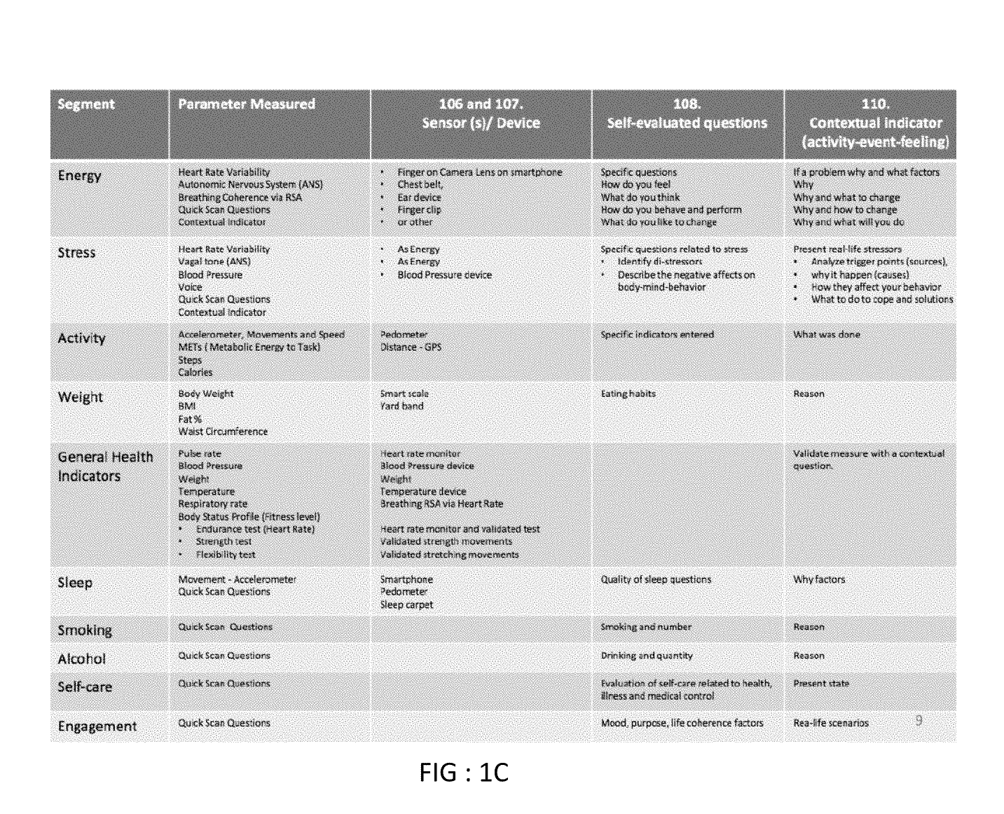

FIGS. 1A and 1B shows schematic representations of system 10 that can perform the self-regulation method 20, according to one aspect of the present invention, and FIG. 1C shows different health segments HS and health parameters HP in a chart. The explanations illustrate an embodiment of system 10 and the self-regulation method 20 that is performed in real-time with activity of the user. In this embodiment, a smartphone 100 operates as the central device with an application 102 performed on the smartphone 100, having access to one or more measurable values 101 that can capture or measure one or more behavioral physiology measures of health parameters HP, for example via internal sensors 106 to the smartphone 100 and external measurement devices 107. Moreover, smartphone 100 is connected to a server 103 via network 104, and the server 103 or the smartphone 100 is also operatively connected to a database 130. The user of smartphone 100 can be prompted or asked, on a regular basis, one or more self-evaluation for activity-event-feel determination questions (AEFDQ) that are correlated with a basic user profile 109A on the smartphone 100 and validated raw data standards, the validated raw data standards being measured health parameters HP that are calibrated or normalized to be put into a specific context.

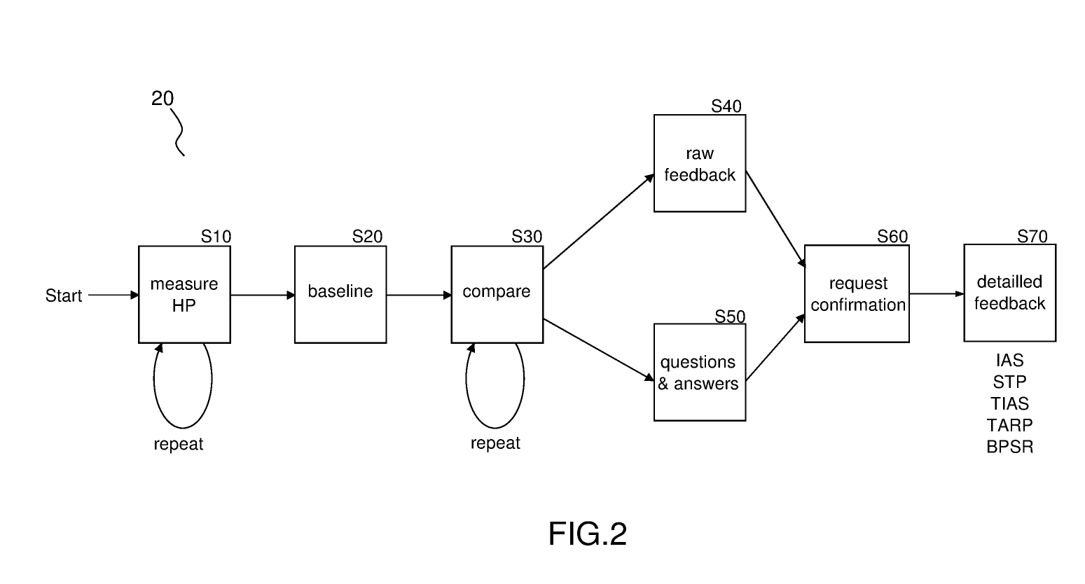

With the system 10, when more parameters need to be analyzed, for example simultaneously measured ones, and more sensitive data is needed, smartphone 100 can connect to cloud server 103 via network 104, to access different cloud-based data and data processing services via server 103 and/or database 130. As non-limiting examples, a full or detailed user profile 112A can be stored on server 103 with database 130 to be accessible via server 103, user baselines 112B can be accessed and updated via server 103, user data can be analyzed with a process 112C that allows to analyze user data based on the measured raw health parameters HP, determine and ask the user AEFDQ questions by a prompt, and the compares the answers to the AEFDQ questions with the raw data, and to provide for raw user feedback and instructions (see steps S40, S50 of FIG. 2), can also be performed via server 103, a user evaluation feedback loop or process 112D can be performed, to confirm patterns of behavior and feedback protocols, and to determine weights and values of different health segments HS. With process 112C, raw user feedback is provided after data analysis, and this raw user feedback can be confirmed by process 112D. With this process 112D, a suggested biofeedback protocol can be applied or trained to the specific user. During the period when a user applies or trains based on the suggested biofeedback protocol, data is recorded and compared with an expected health output, together with a self-efficacy feedback for the user, for example but not limited to a user feedback that indicates that the suggested biofeedback protocol was or is successful, satisfied, or a feedback that indicates a need for more practice, or unsuccessful, poor results. Moreover, data on positive conformation by the user, for example in the form of user likes 112F can be stored and accessed, for example the user confirming his satisfaction with the suggested biofeedback protocol, or a user sharing his positive feedback of one or more aspects of the suggested biofeedback protocol on a social media platform, to stimulate motivation by social media user feedbacks and community feelings. In a variant, data and processes 112A to 112F can be performed, stored, or updated locally on smartphone 100, instead of performing them on server 103.

The system 10 and method 20 can ask for the user for one or more contextual inputs or indictors 110 via a data input interface 111, for example a graphical user interface of smartphone 100, and can display raw data feedback. After an analysis process 113 of the measured data via sensors 101 and historical data, the method 20 can generate instant action solutions (IAS) based at least one of user data analysis method 112C and user evaluation feedback loop 112D, for example by server 103 or also locally in smartphone 100. Next, system 10 is also configured to collect content 120, for example video recordings, audio recordings, voice recordings, and other media data, for example for data analysis on a health status of a user. Also, content 120 can be made available, by authorized access by the user, to a personal doctor, physician, or health professional, via a counseling platform 122, for example by a direct line of communication, for example but not limited to a communication channel in the form of a web chat, video conference interface via network 104, or can also connected to different types of health-related applications, for example but not limited to a nutrition application, exercise application. Also, research labs 123 can be connected via network 104 by smartphone 100, for example via server 103 with inputs from a local or cloud database 130 having video, voice, media, audio, and other content 120 stored thereon that can be made accessible to the counseling platform 122, for example a network-connected computer. With counseling platform 122 a personal doctor or health professional can be given access rights to certain health parameters, for example to change or adapt them, for example in full user profile 112A, and thereafter, the basic user profile 109A on smartphone 100 can be automatically updated. For example, the cloud database 130 can include a database of expert or knowledge advice that has been built by the present method 20, or by external data sources, for specific health and context situations. Also, the software 121 that performs the method 20, either the part running on the smartphone 100, or the part running on the server 103, or both, can be updated upon request or based on scheduled intervals.

In the system 10 and method 20, real-time data of the user can be via behavioral physiological health parameters, analyzed by using date from answers received from the user based self-evaluation of related health to AEFDQ questions 108, and the underlying health parameters. The data is collected via smartphone 100 from internal smartphone sensors 106 and external sensor devices 107, and are thereafter analyzed by a local data analysis process 109 to generate validated raw data standards, for example to determine a baseline for each one of the plurality of health parameters, either at the smartphone 100, at the cloud server 103, or both.

For example, based on the AEFDQ questions 108 that are presented to user, the measured raw data of the health parameters HP can be transformed to provide an adapted feedback. For example, the heart rate HR can be measures as 89, while the heart rate variability HRV is very low, but based on AEFDQ questions 108, system 10 will be provided with contextual information that the user has (a) just recently ended a running activity, (b) along a ten kilometer (10 k) wooden trail, and, (c) has just finished his recuperation mode as the running happened some time ago, for example 10 minutes ago. This new data on context, including past physical activity, running, outdoor, 10 km or more, recuperation mode, is used by the system to compare to the baseline values of the health parameters HP, the system 10 is configured to compare these values with the baseline. With repeated cycles of measuring the health parameters HP, asking feedback in context with the AEFDQ questions, a library of AEFDQ baseline settings is built locally on the smartphone 100 and more complete and extended on the server 103 and database 130, for example by the user evaluation feedback loop 112D and historical data 112E, so that the history of the data can be further compared to an activity related baseline readings, and more accurate health feedback can be provided with continuous use of the system 10 by user.

Data input interface 111 of smartphone 100 can prompt user for answers related to at least one of the actual context of the user, and the actual physical, mental, emotional, and behavioral status to determine a heath index, and this data can be repeatedly analyzed by the local data analysis process 109, for example based on changes of context measured, or changes of one or more health parameters versus the corresponding base line. With an analysis process or method 113, instant action solution (IAS) or suggest training protocol (STP) can determined, refined, and updated, and communicated via the user interface of smartphone 100 to user, based on the AEFDQ questions 108. For example, instructions or suggestions in the form of a message can be displayed on the touchscreen of smartphone 100 of the user. Also, via smartphone 100, the user can consult these suggestions, for example IAS or STP, can confirm them as confirmed actions 115A, or reject them, for example via a displayed button on the graphical user interface of smartphone 100. Next, system 10 or method 20 can generate a new set of suggestions, based on the user feedback of either rejecting or accepting the instructions IAS or STP, to provide a new set of instructions IAS or STP. This allows the user self-regulate by executing or otherwise engaging in the confirmed actions 115A, and or instruct the system to engage in a post measured loop and comparison algorithm or process 117. Comparison process 117 is based on pre-exercise recorded data, monitored values and ranges during the measure and post measure of health parameters HP after ending the exercises, for example exercises that were suggested by system 10 and method 20 in the form of raw feedback, for example baseline information, or detailed feedback of the user.

For example, a breathing exercise to increase heart rate variability HRV under different conditions could have been proposed by the system, for example including baseline, stressor, performance, relaxed breathing at six breaths per minute minutes and back to baseline, each step having it norm value ranges and comparison with previous readings—which is based on an algorithm. If necessary, via the smartphone 100, the user can engage with the counseling platform 122 to engage in remote counseling service with their personal doctor, pharmacists or a health professional, for example using a chat application, a video conference, voice conversation, or to schedule and engage in an in-person interview.

FIG. 1C shows a table with ten (10) health segments HS on health performance, including different health parameters HP that are associated to health performance, different sensors 106, 107 that can measure health parameters that can be indicative of different values of the health segments HS, to measure and analyze the health performance of the user, AEFDQ questions 108 that can be asked to the user to analyze and gather additional feedback from the user on the different health segments, and contextual feedback 110 associated with each health segment that can be provided by the user as answers or additional parameters to the AEFDQ questions 108.

With respect to FIG. 2, a simplified exemplary schematic view of the method 20 is shown for operating the intelligent interactive mobile health system 10, these method steps being part of the system learning (SL). In a step S10 that is repeatedly performed, for example in periodic intervals, the method measures a plurality of health parameters (HP) of the user with one or more sensors 101. For example, the measurements of HP can be done by sensors 101, for example sensors 106 that are part of the smart phone 100, or sensors 107 that are external but in operative connection with smartphone 100, or another type of portable electronic device, including but not limited to heart rate monitor, blood pressure sensor, blood flow measurement sensor, breathing pacer, optical sensors for skin measurements, humidity sensors, sensors for measuring an activity of the nervous system of the user, oximeters, or from external health measurement devices that can communicate with the smartphone, for example but not limited to smoke detectors, image capturing and processing to detect features. In this context, additional signals can be measured by sensors 106, 107 of smartphone 100 that provide for indicators of the present context of the user, including measurements of motion and geographic location including inertial measurement units (IMU) and acceleration sensors, global positioning signal (GPS) receiver sensor, microphone to detect and analyze a voice of the user. While these sensors do not directly measure health parameters HP nor health segments HS, they can provide for measured information on the present context of the user, for example but not limited to detect agitated voice level, detect high or low voice volume, detect state of motion of the user. The health parameters HP that can be measured include at least one of heat rate (HR), heart rate variability (HRV), a blood pressure (BP), a motion activity, and a weight of the user, breathing frequency and intensity (RSA), synchronization between breathing rate and heart rate, different autonomic functions for example autonomic nervous system responses (ANS), sleep quality, smoking activity, alcohol consumption, emotional status, illness, and mind-body coherence. In contrast to the health parameters HP, the health segments HS are more abstract in terms of their physical and/or chemical manifestation, and may not be directly measurable, but can be calculated or estimated based on measurements of one or more health parameters HP, as shown in FIG. 1C. Health segments HS represent indicators for a health status of a user including his physical, mental, emotional, chemical and behavioral status.

Next, with a step S20, a base line dataset is calculated, for example by the smartphone 100 itself or by a data processor that is in operative connection with the smart phone 100, for example a cloud server 103 or networked computing device that can be accessed by the smartphone 100 via a data telephony network 104. A baseline can be calculated for each one of the plurality of health parameters, covering a predetermined period of time, and the baseline can be regularly updated to take into account long term changes of the baselines of the user. The baseline represents an average value of the measured health parameter HP over a given time period, for example a month, that does not or only insignificantly changes when there are temporary spikes or changes to the measured health parameter. The baseline can be regularly updated and calculated based on a time window.

In a step S30, that can be repeatedly performed, for example on a relatively short interval of one hour or less, preferably every five minutes or less, the smartphone 100 itself or by a data processor can compare the different presently or recently measured health parameters with the corresponding baseline. The goal of this step is to detect sudden changes of one or more of the measured health parameters representative of an actual health status of a user, while compare them to the corresponding base line. For example, if the currently or recently measured health parameter exceeds the base line by a certain percentage, the next step S50 of prompting the user can be triggered.

Next, in a step S50, for example upon detecting a sudden change between one or more of the health parameters HP and the corresponding base line, the user can be prompted via a user interface device, for example a graphical user interface that is operated on a touch screen or other display device of the smartphone 100 or other mobile electronic device, to answer contextual questions, the contextual questions related to at least one of physical, mental, emotional, and behavioral status of the user, referred to as activity-event-feel determination questions AEFDQ. The goal is that the user gives feedback or an explanation related to the sudden change of at least one health parameter with respect to the corresponding base line, and this information can later be used to make a detailed analysis of all the health parameters HP of the user, and determine values for the different health segments HS, in a step S70. For example, the user can be prompted to answer questions related to his physical activity, upon a sudden change in heart rate. As another example, the user can be prompted to answer a question related to his emotional well-being or stress level, for example by rating a current stress level he is subject to, upon a sudden change of an increased blood pressure. The prompting can also ask questions about his current mode of transportation, geographic location, or destination he is aiming to visit or render himself. In this step, it is possible that a plurality of questions are answered, for example yes or no questions, questions in which the user needs to rate or give a value to a certain question, and based on the answer, a new question can be asked. This question and answer session that is performed by step S50 can be based on a tree of questions, or questions and answers session that has been designed by an expert in the field of the health parameter in question, for example a doctor, scientist, health expert, physician, as further explained below with respect to step S72 and the decision tree DT. As a result of the questioning and answering of step S50, data becomes available that allows to analyze the impact of changes and trends in the health parameters HP, and their impact on the health segments HS.

In parallel or sequentially before or after step S50, it is possible that the method performs a step S40 of providing raw feedback on the health or health performance of the user. The raw feedback can further include the instructions IAS or STP described above. This step can be repeatedly performed, or a health performance status continuously updated so that the user can, at any time, verify a current raw health status. This can be presented as a value or a bar that represents his overall health performance, for example in a status bar of a graphical user interface of the user. For this purpose, the display device 111 or touch screen of the smartphone 100 can be used to inform the user of the raw feedback. Also, in this step, the validated raw data can be processed, where the raw health data from the health parameters HP is compared to a present context.

In a step S60, the user can make a request to the system to ask for a detailed feedback based on a pre-stored personal basic user profile 109A. The basic user profile 109A can be locally stored on smartphone 100 or on server 103. In this request, the user operates the user interface device, for example an input data screen of a graphical user interface running on the smart phone, to describe his current context, and to request for an activity recommendation given his current context. For example, the user can request the detailed feedback by describing that he is about to engage in a mentally stressful situation, for example an interview or an exam, and that he is currently experiencing a high level of anxiety and a medium level of stress. This can be done by assigning different weights, for example from 1 to 10, for different factors, the exemplary factors being physical stress level, emotional stress level, anxiety level, sadness/happiness level, excitement level, concentration level.

Next, in a step S70, upon the user sending his request to the system 10, a data processing device, for example the smartphone 100 of the user itself, a web or cloud server 103 and cloud storage 130, or a networked data processing device can calculate a detailed health feedback for the user. This detailed health feedback can be based on data of the personal basic user profile 109A, for example age, gender, physiognomy, etc. of the user, based on current and historic base lines of one or more health parameter HP, detailed data of the health segments HS, and data of the current context and historic context situations that have been provided by the user in the step S50 of prompting. Upon calculating and determining the second feedback, the second feedback is provided to the user via the user interface device, for example a graphical user interface 111 of smartphone 100. Preferably, the second feedback is provided with a short time period of making the request in step S60, so that the user can directly engage in a recommended activity pattern proposed by the second feedback.

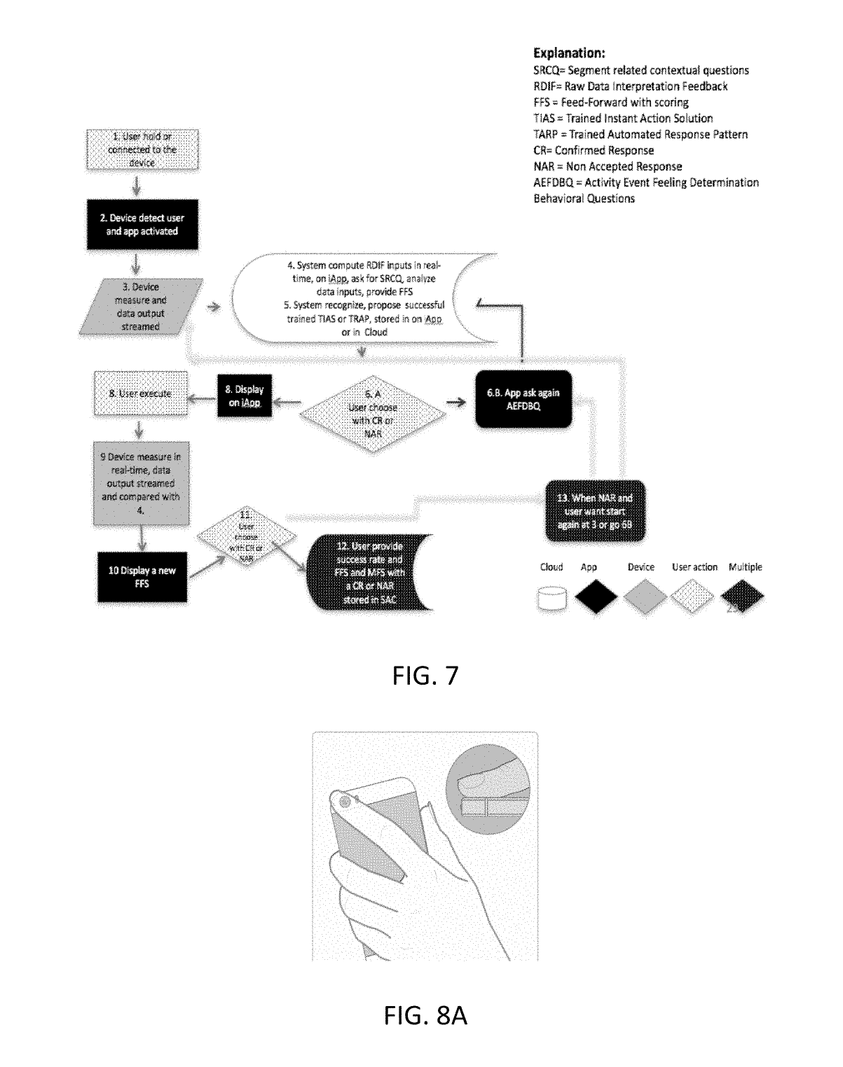

In step S70, the detailed health feedback can initially can include an instant action solution (IAS) that is suggested by the system 10, proposing to the user physical actions of immediate impact on the problem, without to use of any elaborate historical data. For example, this can include to stop physically moving and to engage in a physically calm behavior. Also, the detailed health feedback can also include suggested self-regulation protocols (SSRP) that suggest behavioral patterns to the user, such as mental exercises, meditation, autogenic training, etc. Once the system has been partially or fully trained, it is possible that the detailed health feedback of step S70 includes more complex information for the user. For example, the user can be informed to engage in a trained instant action solution (TIAS), trained automated response patterns (TARP) after using the system 10 for some time, and engage in behavioral physiological self-regulation (BPSR), as exemplarily shown with the flowchart of FIG. 7.

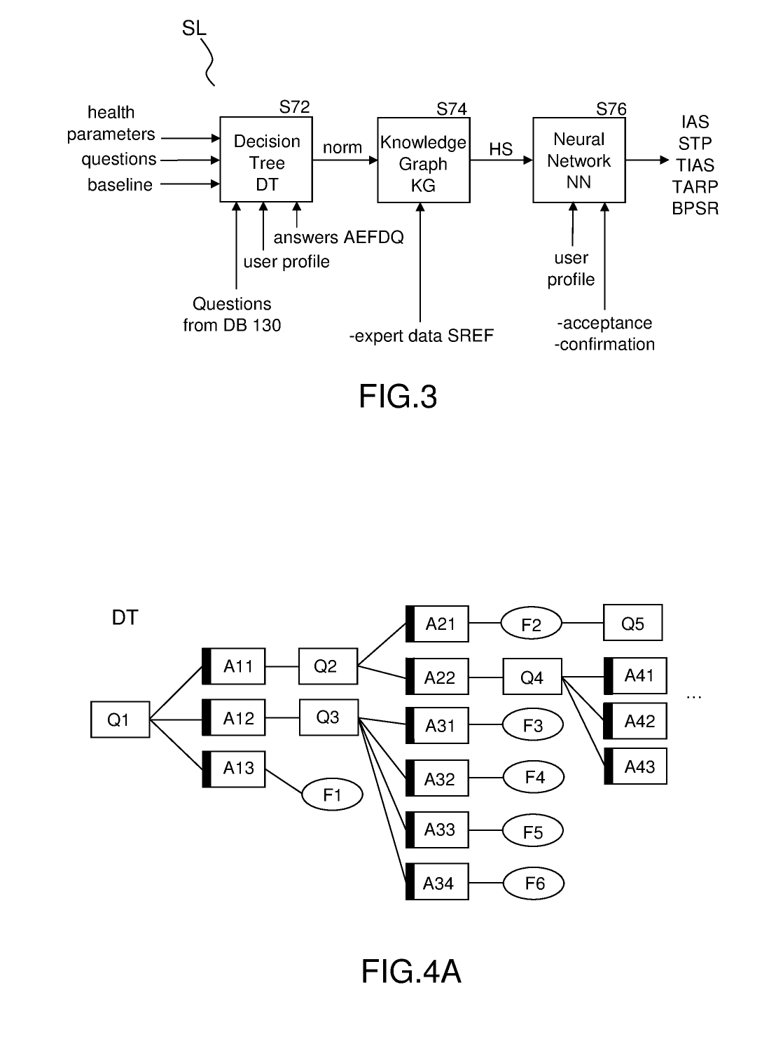

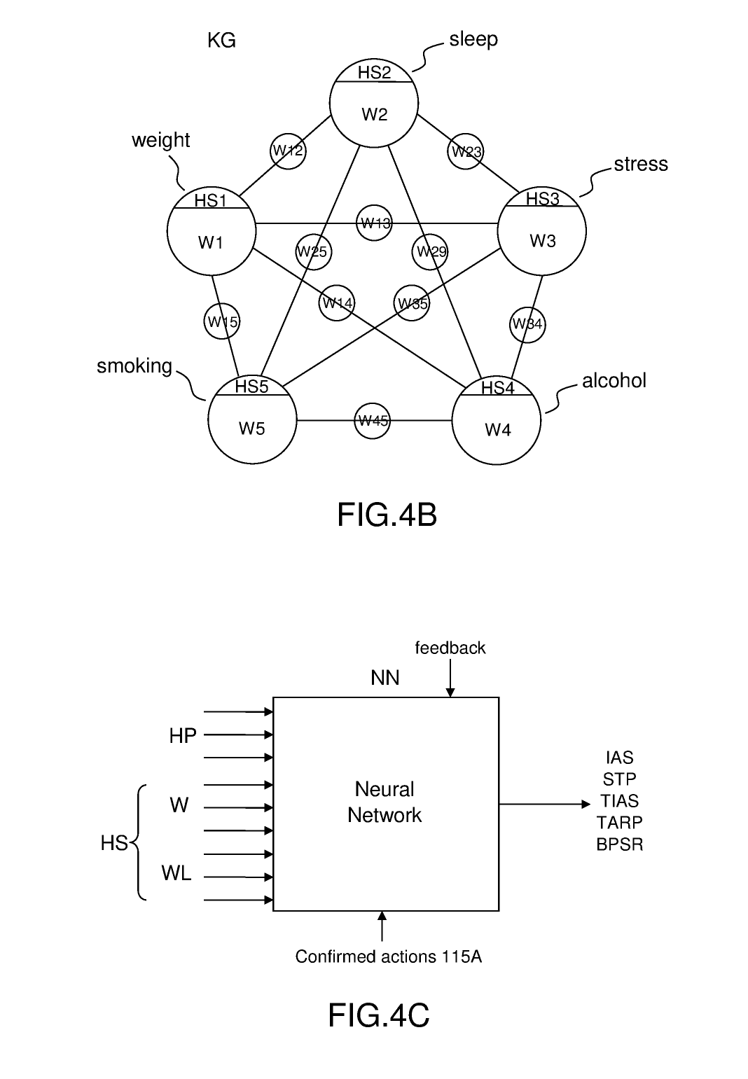

The step of calculating and determining the second feedback S70 itself involves a three-step analysis process, also referred to as the system learning (SL), schematically and exemplarily shown in FIG. 3. This process can be performed on server 103, but it is also possible that the smartphone 100 itself performs these steps. First, the data in the form of answers A are processed by a decision tree DT step S72, to provide for standard normalized values for the health parameters HP, to calculate personalized base line values for the user, and to analyze trends over time. This step involves step S50, in which the user is prompted to provide answers A to one or more questions Q, in certain instances, a specific answer A will cause another question Q to be asked to user. In this step S72, the decision tree DT learning assesses the answers A provided by the user in step S50 and the associated one or more health parameters, to analyze the human health risks, health behaviors, and health measures via sensors in the form of health parameters HP, and as a result, pseudo-intelligence based on normalized values, for example for age, sex, pattern ranges, static responses, personalized base line values, and trends analyze over time can be provided, for example to indicate and analyze whether there is a declining or improving health condition. Decision tree DT can ask a first question Q1 with step S50, and upon receiving an answer A11 or A12, more questions can be asked, for example questions Q2 or Q3, respectively, depending on answer A11 or A12, as shown in FIG. 4A. This can be part of questioning and answering session that is performed in step S50. Depending on the answers received, for example A13 to Q1, or A31, A32, A33, and A34 to Q3, no more questions are asked to the user in this session, and data values are prepared and returned, indicated as F1 to F6. It is possible that the results of the questioning return an ultimate answer or value and no need for more questions of refers again to an optimal result from a previous questioning, the process of the decision tree DT terminates uses the answers as the accurate answer set. Values F1 to F6, are frequency values F that stand in relationship with one or more heath parameters HP, for example heart rate variability HRV, heart rate HR, etc. and can be used to provide for personalized weights, as explained below.

Next, in a step S74, the normalized values of the measured health parameters HP, the personalized baseline values, and the trends of the health parameters HP can be subjected to a knowledge graph KG analysis, as exemplarily shown in FIG. 4B, to determine values and historical data for each one of the health segments HS. This allows to represent or link the measured and historical values of the health parameters HP with corresponding health segments HS. In this analysis by the knowledge graph KG, based on the personal data of the user, including the data of the answers of step S72 and the basic user profile 109A, each one of the health segments HS are weighted with weights W, and at least some of the links between health segments HS are weighted with weights WL. For example, the frequencies F1 to F6 determined in the questions and answers session of step S72 are used to link changes, values and trends of to one or more health parameter HP to one or more health segments HS. This allows to associate each health segment HS with a corresponding weight W, and if a connection can be found between different health segments HS, to weigh a connection between two different health segments HS with a weight WL, or to simply indicate that there is a connection or correlation. This data can then be compared to multiple conditions and measures (MCM) linked to specific related expert feedback (SREF) compared with other MCM parameters and SREF.

According to another aspect of the present invention, the specific related expert feedback (SREF) includes an presentation of at least one of a current state, predicted state, and optimal state that is presented to the user, based on data history and the current values of the health segments HS. This SREF can be designed by an expert. For example, the current state can be presented to user on graphical user interface 111 of smartphone 100, showing one or more health issues that can be related to the health segments HS, for example but not limited to lack of sleep, low HRV, low stress tolerance, difficulties in decision making, short concentration span. Next, a predicted state can be presented to user on graphical user interface 111 of smartphone 100, the predicted state being a state of the user in the future in case there are no changes in behavior of the user, for example to provide a visualization of your body health in a near time future, indicating increased body weight, increased risk of serious health problems and consequences, deteriorated lungs due to smoking. Also, an optimal state can be presented to user on graphical user interface 111 of smartphone 100, the optical state can be combined with an STP, IAS, TIAS and/or TARP, to reach the optimal step, including the indication of a health target for example in the form of a desired value for a health segment HS. For example, the STP can include but are not limited to instructions to take time off work, refresh the nutrition plan, go for a daily long walk, engage in daily yoga, early sleeping hours, meditation, repetition schedules for these actions, engage in exercise, engage in mind-body coherence exercises. Also, the STP can include a schedule for making new requests with step S60 to the system 10 and method 20.

This analysis by knowledge graph KG in step S74 and subsequently by neural network NN in step S76 will enable system 10 and method 20 to generate a second detailed feedback, for example to bring out (1) a targeted prioritized action plan and (2) an overall feedback highlighting insight on underlying causes that is suggested to user to act on, (3) monitoring of effect from suggested action both measuring of psychophysiological parameters like HR, HRV, ANS and different sub-parameters of HRV, for example but not limited to like standard deviation of the heart rate variability (SDNN) and root-mean-square of successive differences of heart rate variability (RMSSD), for example values related to the frequency domain of the autonomic nervous system ANS. Also, with the weighted health segments HG of knowledge graph KG and its history, perceived and present stress symptoms of cardiovascular of physical, mental, emotional and behavioral signs and symptoms can be monitored.

Moreover, the second detailed feedback generated by neural network NN of step S76 can include for example the instant action solution (IAS) providing the user with instructions to act immediately on a short-term issue, an self-training protocol (STP) providing the user with a procedure to be followed over a time period to address a health issue that has been chronic or sustained, a trained instant action solution (TIAS) that will provide the user with a notification or message to execute an already know or trained procedure, and a trained automatic response pattern (TARP) that will provide the user with a notification or message to execute a procedure to be followed over a time period to address a health issue, and a behavioral physiological self-regulation process (BPSR), being a guided breathing protocol for the user to follow via biofeedback protocol and display.

As an example, the health parameters HP can be directly measured in step S10, and will provide for information to determine the status of the different health segments HS, for example the n=10 health segments shown in FIG. 1C. For example, the health segment “stress” of a specific user can be expressed and analyzed by health parameters HP based on measurements of the heart rate (HR), heart rate variability (HRV) as two different health parameters HP, for example by using camera lens of the camera of the smartphone 100 as an internal sensor 106, or via external sensors 107, for example an oximeter put can be put onto one or more fingers of the user, ear-clip, or a chest belt, or a combination of these measurements. Thereafter, data is analyzed and provide a user specific raw feedback can be provided, in step S40. In a step S50, one or more users are asked a set of contextual questions, the AEFDQ questions, related to location, state of mind and body, the user daily living index corresponding to seven (7) specific health parameters HP enabling system based on norm values and recommendations provide an index value.

Based data and the results received from the AEFDQ questions in step S50, updates can be made to basis user profile 109A, and data can be generated to understand, evaluate, and provide data on the following factors: (1) how the specific user perceive stress, (2) what life challenges are part of the life of the user during a period of time, for example the last 12 months, (3) what stress symptoms the user has experienced, intensity and pathways like cardiovascular, muscular, mental, immune reaction, etc. (4) what are the related specific stress response of the user, for example but not limited to panic, worries, overeating, anxieties, etc.

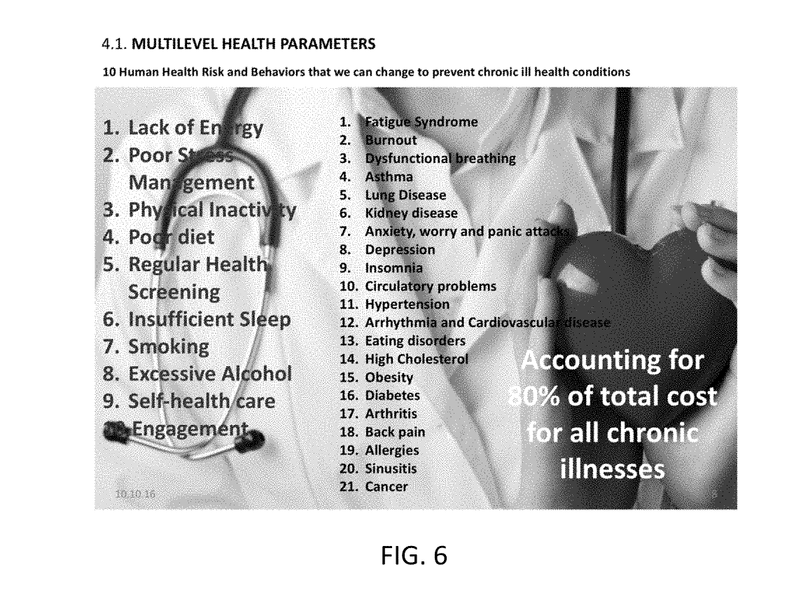

As part of the decision tree DT of step S72, with expert feedback, for example data and statistical results that was previously provided inputted by a doctor, physician, or a health professional, herein referred to as SREF, the knowledge graph KG step S74 is established and refined, to provide for additional data that influences weights W of health parameters HP1 to HPn and weights WL that interlink the health parameters HP1 to HPn to each other, as exemplarily shown in FIG. 4B showing a graph with five (5) health parameters, showing weight W to each health parameter, and a weight WL to each relationship between each health parameter. Also, between some HP there may be no connection at all, with WL=0. Typically, the graph includes around ten nodes for ten different health parameters HP, as represented in FIG. 6. The weights W and WL are generated based answers to AEFDQ questions, basic user profile 109A, and expert knowledge, for example SREF, that is made available to system 10. Thereafter, the method 20 determines the most accurate solution for the user, for example by providing trained instant action solution (TIAS) with detailed feedback in step S70, by using a neural network NN in a step S76 that processes the data from the knowledge graph KG obtained in step S74, as shown exemplarily in FIG. 4C. Additional influence of the neural network NN is obtained by user acceptance with confirmed actions 115A, and after monitor the execution process with a self-efficacy feedback from the user. This allows the user to train the algorithm of system learning SL presented by steps S72, S74, S76, and once the user have trained the solution over time, the interaction of the user with the system 10 and method 20 can become a TARP that is based on a suggested specific training protocol (STP) by the system 10 and method 20 that is applied to a specific measured context that user have “confirmed”, reported “like” and “obtained a high self-efficacy score” after each training and overtime as the system suggest the same. STP and training performed by the user with success then becomes a TARP. Once it has become a TARP, system 10 and method 20 just provide the solution and the user engages in specific activities and training to execute. Overtime, activities and training by the user become fully natural self-regulation response, and can become behavioral physiological self-regulation (BPSR). BPSR is similar to TARP, but it takes more time for the user to learn and describes a self-therapy learning to overcome an emotional or physiological barrier to engage in a health improving activity, for example a phobia, fear, aversion, loathing with respect to a specific activity and/or learning.

As discussed above, the neural network NN that receives the data from the knowledge graph KG can be specifically trained for a user and the corresponding user profile 109A and/or 112, to achieve an optimal function. For example, one training function for the neural network NN to achieve is a proposed STP that has been linked to data of the user profile, base line information, specific actual context, and changes in health parameters HP, for example determined by a health expert. As another example, another training function for the neural network NN to achieve is a proposed TARP that has been linked to data of previously successful executed STP by the user, the user profile, base line information, specific actual context, and changes in health parameters HP, for example determined by the feedback of the user related to the STP, by confirmed actions 115A.

With step S50 and AEFDQ questions proposed by system 10 and method 20, and the answers provided by the user allows to enrich the basic user profile 109A and the full user profile 112A with multiple health lifestyle and behaviors indicators by answering the contextual questions that were presented to the user in step S50, and by providing automated updates to the one or more health parameters, and their associated baseline, the knowledge graph KG that links and gives weights W to all the health parameters and weights WL between two health parameters gradually evolves, and the parameters and nodes of the neural network NN of step S76 measures following the knowledge graph KG pathways gradually the parameters and nodes of the neural network NN and a library of parameters are constantly upgraded and analyses based on rules and norm values enabling system to detect early maladaptive patterns and instantly provide proven solutions to help user to trigger a TARP.

According to another aspect of the present invention, the unique combination of the features of the present system 10 and method 20 provides for the ability to take multiple health lifestyle and behavioral indicators from various sources, for a specific user.

The analysis of the data from the knowledge graph KG by the neural network NN in step S76 brings all the actual and historic data together, to learn to evaluate the user in a more holistic integrated approach and specifically with the global solution as it prime focus. The global solution is connecting human health risks, for example the ten (10) human health risks shown in FIG. 6, and behaviors to the user profile and link all stored data, solutions and level of self-efficacy in doing them to become a TARP.

Moreover, system 10 and method 20 can document and categorize different actions and responses in a range of situation and suggested instant action (IAS) or suggested training protocol (STP) to a psychophysiological response parameter, for example but not limited to HRV, ANS, Breathing pattern,), context and interrelated factors (CIF), such as environment, personal physical, mental, emotional and behavioral state, recorded solution outcome (SO) of a post measured response, and self-efficacy ratio to train system to generated accurate specific IAS-STP based on highest self-efficacy ratio that is determined from the knowledge based parameter, CIF, and SO to train SARP and SBPSR that gradually becomes a TARP and TBRP.

Moreover, the system 20 can detect early a health risk, abnormal heath pattern or maladaptive health. Also, the system 20 can be configure to alert the user based on specific situations. A TARP can be generated and presented to the user for which a response is known, a new unknown response can be analyzed to understand what to suggest and monitor self-efficacy execution. Gradually, after some learning by the knowledge graph KG and the neural network NN in steps S74 and S76, gradually the user can build up a library of TARP's in his user profile, for example the full user profile 112A that are ready to interact via different situation or conditions. Moreover, with the learning, the system will be able to propose additional global solutions that will directly interact with the underlying cause. This is a typical problem in classic healthcare where things are fixed that can be seen, but problems or issues that cannot be seen are not fixed. According to another aspect of the present invention, the system 10 and method 10 is thought to grow and improve along with the continued use of a user, the decision tree based-algorithm in step S72 starts by using expert knowledge and population data, and evolve by using more and more the user profile.

According to another aspect of the present invention, the system 10 and method 20 can, over time, detect early risk factors based on indicative values but also detect early underlying feelings and behaviors that is not effecting human health parameters HP. This is one of the key advantages of the present system 10 and method 20, as it can take into account multiple health lifestyle behavioral parameters starting with a questionnaire, for example in step S50, measured health parameters, provide feedback based on contextual interrelated factors to bring out a solution that the user can train and apply with self-efficacy feedback. Also, the system 10 is constantly updated and over a lifetime it will enable user to live a more healthily, longer and better quality of life.

Moreover, according to another aspect of the present invention, the user profile, for example the full user profile 112A stored at the server 103 can become central in both ways as of (1) training the user to anticipate early health risks based on history data generated detailed feedback of the plurality of health parameters HP, and (2) monitor the ability of the user to bring his health back to normal or via additional expert inputs, as new contextual questions or new health parameters HP can be added into the decision tree DT of step S72, and the knowledge graph KG for weighing health segments HS with weights, based on the information gathered of the health parameters HP, and weighing and indicating relationships between health segments HS with weights WL, designed to provide for information on specific better recovery related to identified new health parameters need, and obtain additional personalized solutions thought the simulation by neural network NN to adapt the training to a new context to minimize the health risk and maximize a specific wanted health outcome.

Basically, with the features of system 10 and method 20, new needs can be anticipated and predicted, to new contexts based on measured early health risks signs and indicators, and adapt the needs based on full user profile 112A and the history data of the full user profile, and add new expert knowledge that can be provided on a regular basis, in order to have the user profile evolve, assisted also by external health expert inputs for a new identified specific need that after will become part of the automated system 10 and method 20.

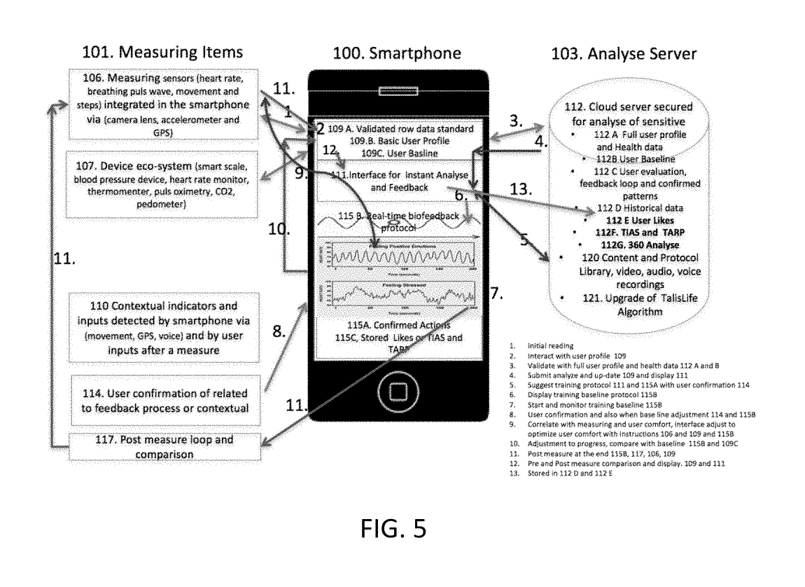

FIG. 5 schematically shows the system in a user wants to download a real-time biofeedback training (BT), with FIG. 6 showing different health segments and the health risks and behaviors and behaviors associated thereto. When at least some data on all (10) ten health segments of the human health risks and behaviors has been collected, it is possible to perform a global analysis by system 10 and method 20. The collected data and information as data on the health parameters can be stored both at the smartphone 100, for example as dashboard information 113 or logbook 115, and can also be stored at cloud server 103 and the associated database 130, for example as user-specific historical data 112R and user feedback and likes 112F, as well as TIAS or TARP 112G. Server 103 with an associated back-end cloud and database 130 can provide for a more in-depth global approach and over time also take into account additional accumulated data in the historical data 112E and user likes 112F user likes to adapt certain biofeedback training (BT) and improve choice of IAS and STP 119B and use suggested global challenges to optimize effectiveness and choice of self-regulation protocols SRP can be downloaded from server database 130 to the application of the smartphone 100 and global intelligent logbook 119C intelligent 360 logbook. These protocols are named suggested self-regulation training protocols (SSRTP).

According to another aspect of the present invention, with the system 10 and the method 20 it is also possible to determine when a SSRTP has been identified as an IAS or STP in relation to a most effective self-regulation response of the user during an interactive recorded BT session. This type of SSRTP for the user can eventually become a trained instant action solution (TIAS) or a TARP and stored both in the logbook 115C at the smartphone 100 and the TIAS or TARP 112G.

When the user is connected or otherwise subject to measurement by a sensor 101, for example via an internal sensor 106 or an external device 107, and smartphone 100 measures and captures underlying health parameters HP, the system 10 can alert the user with smartphone 100, for example by a trigger, a vibration alert, a message prompt, a sign or symbol on the screen, or a sound or a voice alerting the user who recognize the combination and trigger to call into action the related TIAS/TARP, so that the self-regulation loop is completed.

FIG. 1B schematically shows a multilevel intelligent mobile health system 10 for behavioral-physiological self-regulation. In the example shown, the multilevel intelligent mobile health system measures several, for example ten (10) health parameters HP in real-time for behavioral-physiological self-regulation to monitor human health risk and behaviors that are expressed as health segments HS, to detect in a person both healthy habits and unhealthy habits via a (1) self-care inventory (questions and answers session, questionnaire), 2) behavioral—physiological stress profile measuring HR, HRV and ANS and (3) daily living index (contextual question after the behavioral-physiological stress profile, on level of stress, fatigue, lack of energy, dysfunctional breathing, early burnout signs, blood pressure status, activity level and weight, with additional help of measure specific AEFDQ to propose personalized instant action solutions (IAS) for acting immediate on a problem, or suggested training protocols (STP) to re-train identified unhealthy patterns to become gradually as being repeated healthy Trained IAS (TIAS) or Trained Automated Response Patterns (TARP).

The system 10 and method 20, with the portable user device in the form of a smartphone or tablet 100 interacts with one or more external health devices 106, 107, for example external devices 107 that can communicate wirelessly via Bluetooth, an application software 102 that is interacting with the user on the smartphone or tablet 100, and with a cloud database 130 and cloud server 103 that can analyze comprehensive and sensitive data and can partly be downloaded to the smartphone or tablet 100 for data updates and for off-line use.

The system 10 and method 20 can measure, display in real-time on the smartphone screen (1) measured health parameters HP with sensors 106/107, (2) analyze with user profile 109A and validated raw data standards, for example base lines, first from the application software and then updated from cloud 130 in the form of cloud data 112A to 112C, (3) display raw data by graphical user interface 111 to user, (4) interactive with user by a questions and answer session with AEFDQ questions 108, (5) analyze these responses again on the application software 102 or by cloud server 103 if needed, the measured health parameters HP to determine status and the weighted health segments HS to provide user-specific feedback, (6) system 10 and method 20 request after feedback a contextual input 110 from user via one or more questions, or detect also via sensor, for example (7) with GPS sensor as an internal senor 106 to smartphone 100, or accelerometer sensor of smartphone 100, (8) and compare with user profile 109A, and (9) previously measured health parameters HP 112D that can be accessed from cloud server 130, (10) and compare with history data 112E, (11) and also compare with user confirmation or acceptance actions in the form of “likes” or positive feedback 112F of IAS or STP before providing (12) personalized second detailed feedback 113, for example feed forward (FF), for example with suggested IAS and STP that (13) the user after consultation 114 can confirm, for example by a confirmation response 115A, (14) if not confirmed a new IAS or STP as a second detailed feedback can be suggested by system 10 and method 20 until user (15) accepts to do it, the IAS or STP is displayed on graphical user interface 111 of smartphone 100. System 10 and method 20 can receive from user a confirmation by a conformation response or confirmed actions 115A, can perform the real-time biofeedback protocol 115B where you see your presently measured data of one or more health parameters HP on the graphical user interface 111, for example as an evolving curve, and can perform and show data of stored logbook 115C from corresponding TIAS or TARP. Moreover, the system 10 and method 20 can perform other types of method steps on an optional basis, for (16) example a post measurement process 117 analyze a progress of the user, and to make comparisons, (18) perform updates to application software 102 of smartphone 100 based on cloud data, and (19) and when data on all analyzed health segments HS, for example all ten (10) health segments HS are completed or a satisfactory amount of data has been gathered, to provide for an aggregated global view or health index 119 of the user, taking into account all health segments HS, (20) with personalized second detailed feedback that is displayed on the graphical user interface 111 of the smartphone 100, (21) user confirmed response 114 or not, and (22) system 10 and method 20 identify and deliver IAS or STP and also SSRP from already confirmed actions 115 by user and stored on APP or analyzed and downloaded from cloud server 103 or cloud storage 130.

With system 10 and method 20, after a certain number of measurements of health parameters HP, for example a minimum of seven (7) measurements and with feedback from the user by confirmations over time, the software application 102 of smartphone 100 can download from cloud 112 via network 104 user likes IAS or STP's, and SSRP gradually from using them becomes recognized by the system, 112G or 115C, linked to/with a specific situation based on measured self-regulation score in a learning context, as TIAS or TARP, when appear again trigger an user alert on smartphone 100 or on device by a voice sound, vibration or light or an icon.

According to another aspect of the present invention, the system 10 and method 20 can measure via smartphone-internal sensors 106 or external device 107 the fatigue via the heart rate variability (HRV) and breathing pacer (RSA). When both in the synchronization and the frequency is high, this serves as an indicator of good general health, when these two values are out of sync having a low frequency, this can indicate unhealthy functions or pattern of the body of the user. This data can be compared with the basic user profile 109A and the validated row data standards 109B, and with data of full user profile 112A and other user data 112B, 112C, and 112D that is stored on database 130 to determine if a maladaptive unhealthy pattern or behavior is present, processed by an alert indicator 125 that trigger a user alert that can be displayed or otherwise communicated to user, for example via graphical user interface 111 of smartphone 100.

Next, system 10 and method 20 can initiate an appropriate real-time biofeedback self-regulation training protocol SSRTP 115B displayed on the graphical user interface 111 of smartphone 100, the most adapted protocol 115B, the protocol 115B able to be stored locally at the smartphone 100 and updated with data from cloud 112D in the segment of behavioral physiological respiratory stress profile for measuring in real-time the heart rate (HR) with sensors 106, 107, to measure with the ability to extract HRV, RSA and ANS via camera lens (as sensor 107) or heart rate monitor device (as sensor 107) with similar ability to extract HRV, RSA and ANS. Moreover, with a breathing pacer as a SSRTP 115B, it is possible that the user gradually match his present breathing rate measured via sensor 106 with the one given by the breathing pacer, for example by a breathing cursor that is correlated with the breathing wave (RSA) in conjunction with HRV, and compared 109C data inputs 109C from base line to what user can be adapted to, and that the user can obtain an increased Hz frequency of HRV and RSA wave to reach a full synchronization with a high self-efficacy rating. This is a loop that can be performed between sensors 106/107, for example camera or other device inputs, user baseline 109 representing his comfort zone, raw data feedback interface, provided by step S40, this data shown on the graphical user interface 111 raw data feedback interface and adaptation to SSRTP 115B and user confirmation 114.

The behavioral-physiology stress profile training protocol can be conducted over eight (8) minutes with the following stages:

Stage 1 (2 minutes) a present measure is taken with sensors 106/107, the user is asked for contextual indicators are asked 110, Stage 2 (2 minute) sensors 106/107 including breathing pacer 115B are adapted to user present comfort zone shown in the baseline measure that is 114 confirmed by the user, system start recording 106/107 in real-time HR, HRV, ANS. RSA; and correlated with user profile 109 and graphical user interface 111 on interface for instruction and raw data feedback.

Stage 3 (2 minutes) Via graphical user interface 111, the user can be instructed to respond to three (3) challenges (stressors), follow the breathing pacer as SSRTP 115B which adapt to user pace comparing recorded data from sensors 106/107 with voice guidance 110 according to measured parameters performance and to adjust, be in control with breathing pacer as an SSRTP 115B displayed and adapted in real-time on graphical user interface 111 to guide the user to best self-regulate. Data on the three (3) challenges are recorded, analyzed and on shown on graphical user interface 111 and compared with previous measures stored in the user baseline109C. User can be instructed of the details of these challenges by a voice message, video message, audiovisual message, text message, etc. by smartphone 100.

Stage 4 (2 minutes) recuperation phase where user is guided by a relaxing voice, or preferred music or picture following the breathing pacer as an SSRTP 115B which gradually adapt the users to maximal comfort of breathing wave of real-time measure 106 or 107 HRV, ANS and RSA. The system record the physiological parameters (HR, HRV, ANS and RSA), continue to adapt the breathing pacer to bring the user to an optimal behavioral physiological pattern for the user to confirm actions 114, system 113 analyze at the end, display result and suggest related actions 114 that user 114 can confirm or contextual question are asked and another suggestion will appear.

Next, the user can perform a behavioral—physiology stress profile or similar protocol with the system 10 and method 20, and the system 10 and method 20 can automatically initiate the breathing pacer as the SSRTP 115B to the baseline and guide the user within that same parameter or stretch for optimal training.

The process is an interaction between measuring sensors and devices 106/107, user profile 109A, and the baselines, for example the validated raw data standards 109B, and the graphical user interface 111 of smartphone 100 can display the raw data feedback, AEFDQ questions 108, and contextualized input data 110, to process and display 113 full analyze and suggestion to be confirmed by the user with action confirmation 114 and if needed adaptation of suggestions by the system 10 and method 20.

Before using system 10 and method 20 for a daily routine, the user can first perform a method for initializing and configuring the system 10 for the specific user. In this respect, first, the user can run a first time configuration protocol with a seven (7) day baseline recording period in the morning just after wake up involving Stage 1, a (2) minute resting stress check using camera lens as measuring sensor 106 with Stage 2, continue with a two (2) minute paced breathing and ending with Stage 3, seven (7) contextual questions related past days daily living on perceived present stress level, perceived general feeling, last night sleep, the physical activity of the previous day, food intake, alcohol and practice of mindfulness/mind body coherence training.

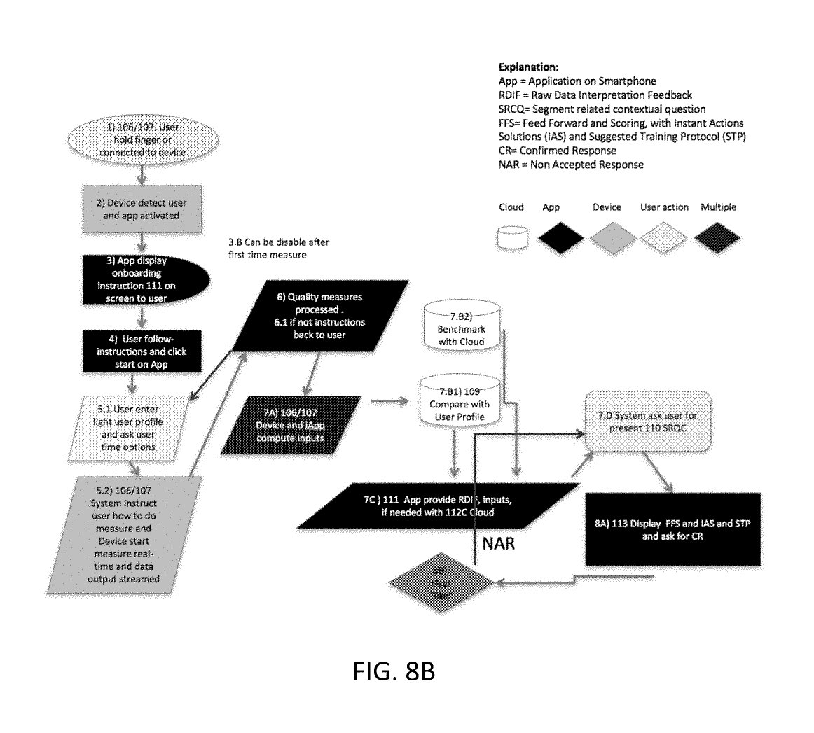

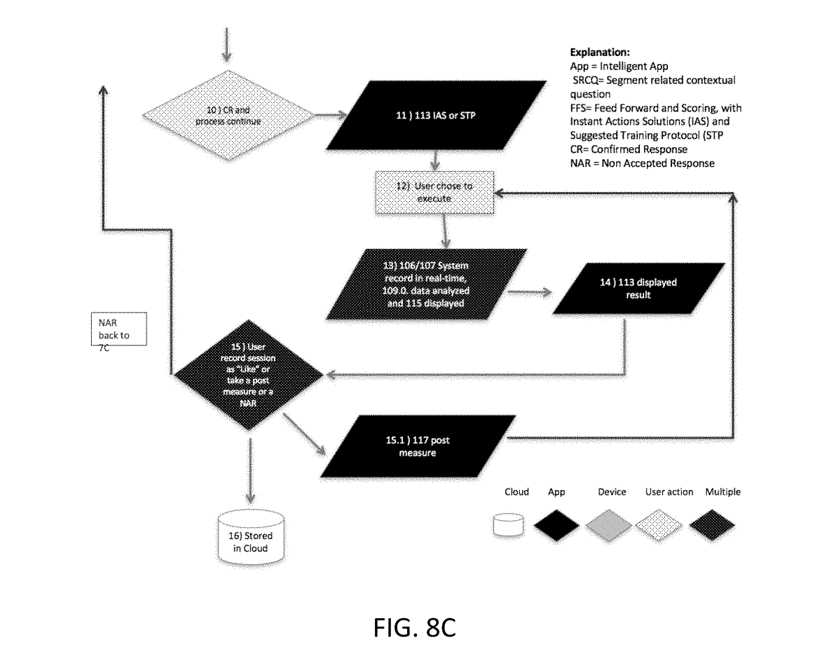

FIGS. 8A, 8B, and 8C schematically show the establishment of the first-time parameters (FTP) for a user, with the first-time self-regulation (FT-SR) protocol or process. The method includes a step (1) where the user holds finger on camera lens as external device 106 of smartphone 100 or is connected to an external HR monitor 107 or other device (FIG. 8A), the device connected to the application software 102 on smartphone 100, for example via a wireless interface such as Bluetooth, a step (2) where sensor devices 106/107 detect the user and activate application software 102 of smartphone 100, a step (3) where the application software 102 instructs user via the graphical user interface 111, in a step (4) the graphical user interface 111 gives user instructions 114 and click start, step (5) application software 102 instructs sensors 106/107 to measure health parameters HP, and the measured data is recorded, step (6) quality measures are applied to the measured data, to see if the data valid for further use and processing, system 10 and method 20 will require another measurement of health parameters HP or generate a user notification that the signals were poor and insufficient for further processing. Step (7A) where data of the health parameters HP and the user profile 109A is analyzed in real-time, and step (7B1) Resulting data is compared to benchmarks related to the user profile 109A, step (7B2) the data can be benchmarked and stored in cloud 103, 130 if requested or needed, step (7C) raw data interpretation feedback (RDIF) is generated, for example by establishing standard values and baseline comparisons, for example each day during a week, in step (7D), on a regular basis, the data is backed-up to the cloud 103, 130, in step (7D) raw feedback scoring is calculated and displayed with questions that are related to the health segments, as contextual questions, to confirm accuracy of the response CR of the user. Step (8A) calculation and display of the second detailed feedback on graphical user interface 111 of smartphone 100, for example a scoring and feed forward (SFF), step (8B) in which the user can confirm his satisfaction with the second detailed feedback, for example by user likes 112E, step (9) another SFF provided by system 10 and method 20, and step (10) when the user confirms actions by CR, the process continues, or if user did not accept the second detailed feedback, system 10 and method 20 will generate an invalid second detailed feedback, and a new analysis period starts, for example a seven (7) day process.

User have provided a CR, an instant action solution (IAS) 113, or a suggest training protocol (STP) can be proposed as second detailed feedback and can be chosen by user in a step (12), (13) system 10 and method 20 record in real-time and 115 display figures with recommendations and (14) after provide post result displayed on 113, if wanted a (15) user can save session as a “like” or with NAR take another post measure back to step (7D) to restart the measuring loop with a new added 108 SRCQ or if quality signal not good restart at step (12) or when CR like continue 16 with update and stored in cloud server 103 or cloud database 130.

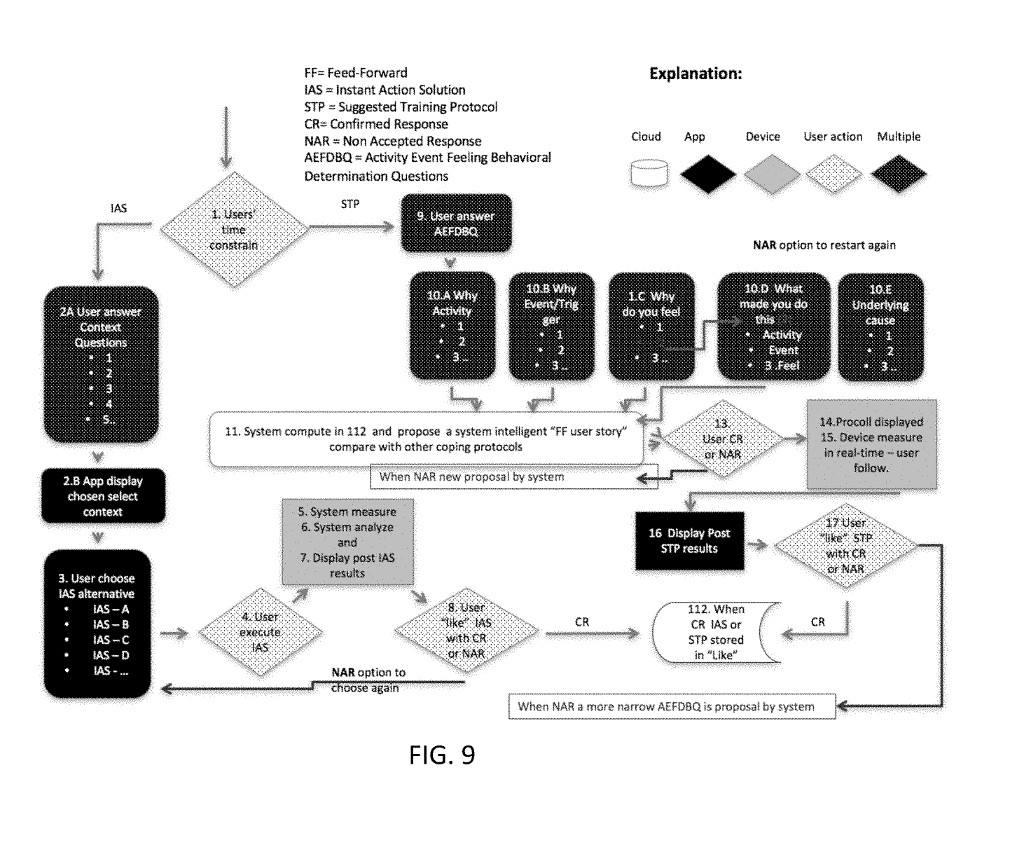

Next, a step of early health problem indicator and self-regulation is explained, with respect to the flowchart shown in FIG. 9. When user experience unhealthy situations, an early health problem indicator, a first time user of system 10 is configured identify learned unhealthy behavioral physiology responses, or system 10 is configured to detect a shift in a health state, the system 10 can also determinate a first time appropriate intervention by assessing HR/HRV/ANS/RSA measured via internal sensor 106 using camera lens on smartphone 100 or an external heart rate monitor as external sensor 107, stream and collect in real-time data, evaluated first the application 102 of the smartphone 100, and then transmitted to the cloud database 130 for a scan of multiple parameters and before displaying a RoDIB analyze 111 on the application software and after request from user: Step (1) If a time constrain less than three (3) minutes system propose a quick IAS with select number of various protocol alternatives, step (2) if there is a need for a full consulting, system 10 proposes a in depth AEFDQ questioning to analyze, after the data are analyzed by cloud server 130 runs another a full scan analyze of the full user profile, baseline, stored user data, user evaluation, historical data, user likes and TIAS and TARP to propose a specific system intelligent biofeedback guided protocol.

Moreover, the integrated behavioral physiological system can propose second detailed feedback to the user to retrain unhealthy patterns by offering user two or more different options in relation to retrain suggestions the via IAS or STP.

For example, when choosing IAS: Step (1) 108 time constrains, a choice of the user, the user choose IAS step (2A) user answer 110 to contextual questions, step (2B) application software to analyze the inputs and compare with confirmed actions 115 and user likes 112F stored in server 130 and 113 display selected IAS alternatives, step (3) user choose an IAS alternative, step (4) 115 user execute step (5) system measure using one or more measures via sensors 106/107, step (6) 109 stream data to app for analyze and step (7) 113 display post FFSAS. Step (8) User like session with CR or NAR, when CR stored in 115C on the application software 112F in cloud server 130. If NAR system re-start at step (3) again.

Next, when choosing STP, system 10 and method 20 can perform the following steps: step (1) 108 time constrains, a choice of the user, the user choose STP, step (9) by first answer AEFDQ questions 108, and a short route cause analyses on step (10A) Why this Activity 1, 2, 3, step (10B) Why this Event 1, 2, 3, step (10C) Why do you feel 1, 2, 3, step (10D) What made you do this Activity, Event, trigger that Feeling, 10E Underlying causes the Activity, Event, Feeling—all with drop box alternative—if not enough personalized inputs that system can recognize. Step (11) System compute in Cloud 112—a scan of all parameters and step (12) propose a system intelligent guided biofeedback protocol, (13) user confirm or NAR, (14) Protocol displayed in 115, (15) Selected device measure with sensors 106/107, (16) Display post measure 113 with FFSAS, 179 User confirm “Like”.

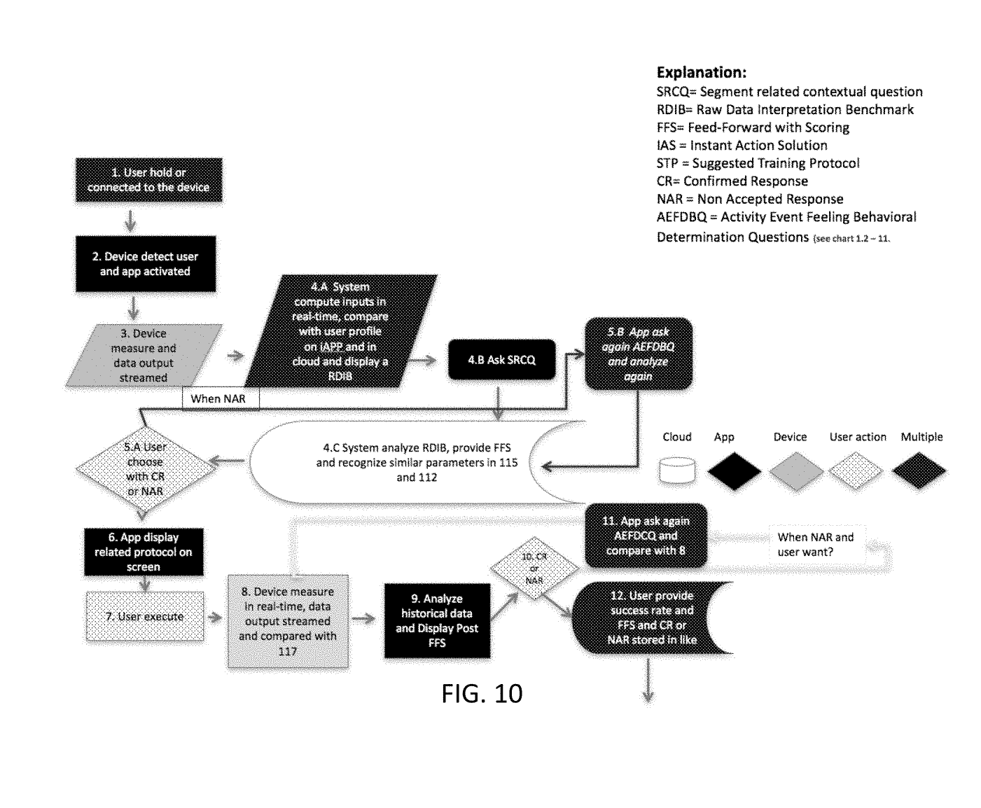

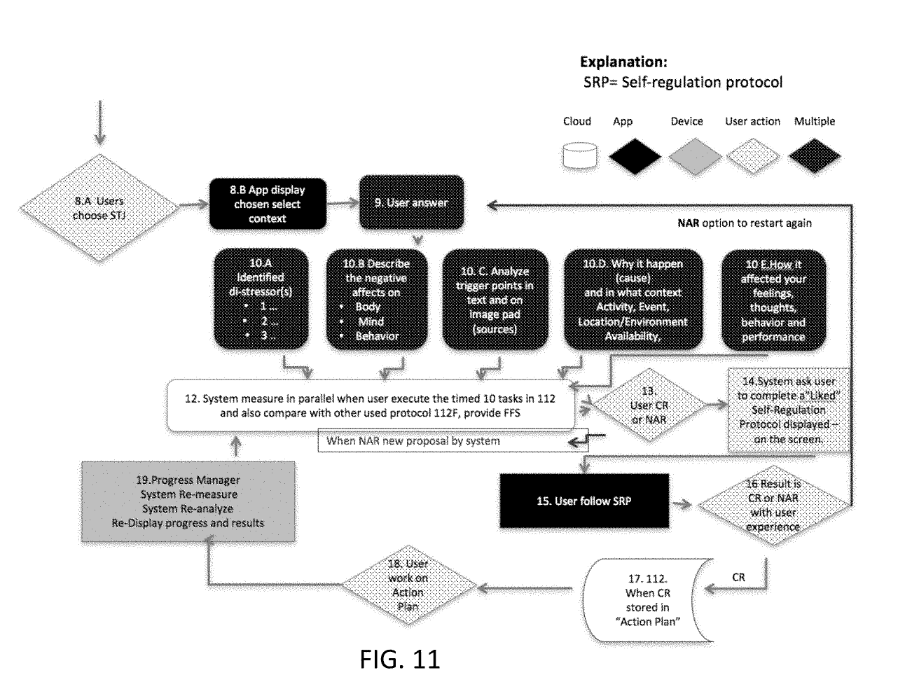

Next, a step of the method is discussed in which a similar event category happens again, in which system 10 can recognize behavioral physiological parameters (SRBPP), schematically represented with flowcharts in FIGS. 10 and 11.

For example, the user feel pressure or need to refuel energy or increased blood pressure, step (1) hold or connect to the device 106, step (2) system is activated, step (3) measures, step (4) system compute inputs in real-time, ask for Segment related contextual questions (SRCQ), analyze, provide a RDIB, 5) system recognize similar physiological parameters select stored in 115C or 112 likes, and choose a related IAS/TIAS or STP/TARP proposed to the user and to be confirmed. Step (7) System display on the Smartphone screen the selected action or protocol, step (8) user execute, step (9) system measure and at the end 117 compare with a pre-post and (10) analyze with historical data in 112, before displaying second detailed feedback on graphical user interface 111 of application software 102. Step (11) User consult by CR or NAR, if NAR back to step 10. (12) System reinforce IAS/TIAS or STP/TARP when good in the “Like Library” on the Smartphone 115 and in cloud 112, (13) if NAR and user want process restart at step (9), or a re-start at step (6).

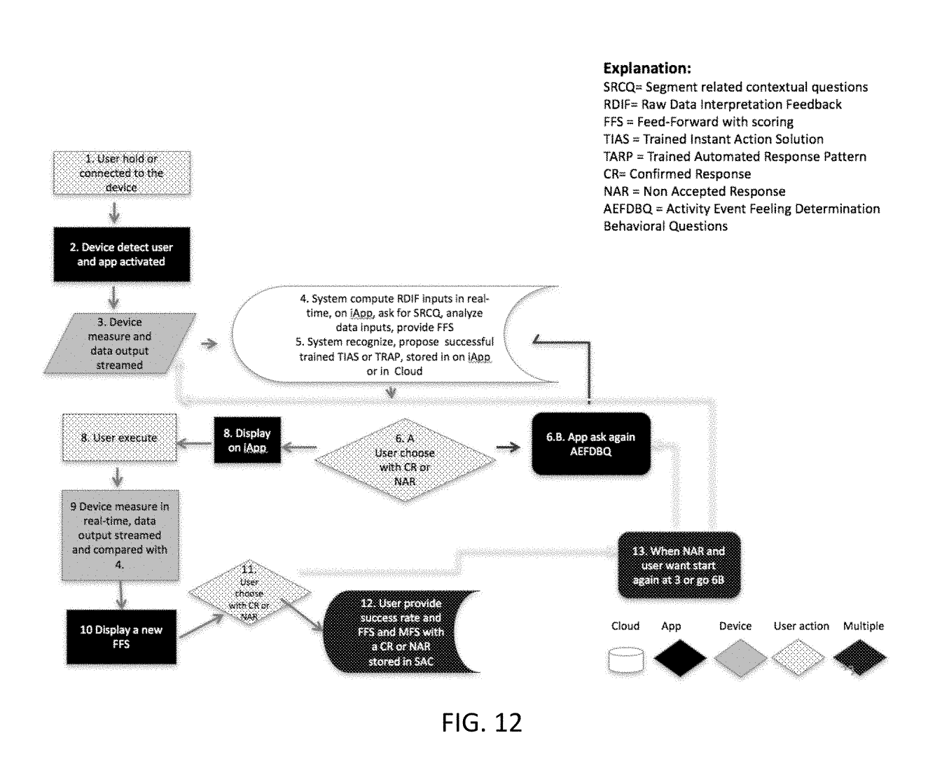

Moreover, overtime system intelligent recognize similar event category when it happens again, to perform a System Recognize Behavioral Physiological Parameters (SRBPP). This part of the method is schematically shown in the flowchart of FIG. 12. The artificial intelligence of system 10 and method 20 will overtime recognize similar events. Step (1) User hold or connected to device, step (2) system is activated, step (3) sensor or device measures, step (4) system compute RDIF inputs in real-time with the application software of smartphone 100, ask for PCA inputs, analyze and after provide first instant feedback FFSAS, step (5) and recognize and automatically propose user a successful trained TIAS or TARP stored on application software 115C or in cloud server 130 for download 112F, step/6A) before execution or download user CR or NAR, step (6B) when NAR additional AEFDQ and back to step (5), step (7) system display with graphical user interface 111 of smartphone 100 the selected TIAS or TARP and step (8) user execute, step (8) real-time protocol and system measure and step (9) at the end provide 117 pre and post scoring, and a step (10) a new analysis to provide for second detailed feedback by application software of smartphone 100. Step (11) User satisfied by CR, step (12) reinforce the TIAS or TARP evaluation with a rating to on PCA and self-efficacy, stored in cloud server 130 when good stored in likes (115C in application software or 112F in cloud server), step (13) when NAR start over again or additional AEFDBQs from 5B. User will train the same TIAS or TARP related to a recognized event and over time it will become a TARP linked to a specific sound from the smartphone, a light combination on the device, an image on the smartphone screen or voice alert on smartphone that user will learn and recall next time.

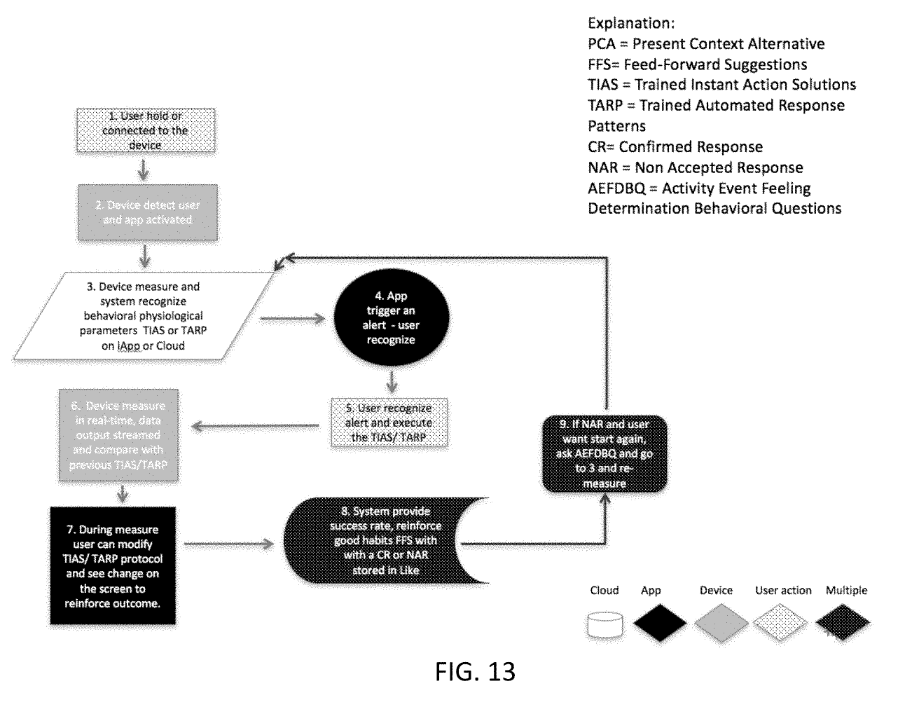

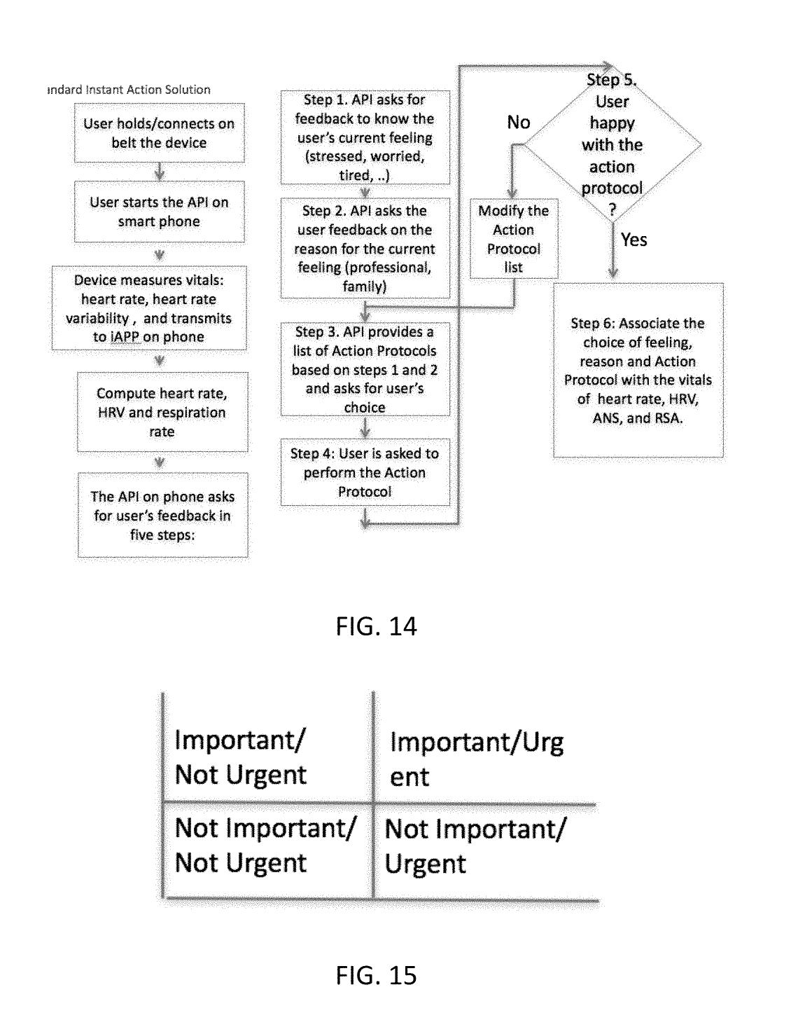

Next, TARP can be used for behavioral physiological self-regulation, as schematically shown in the flowchart of FIG. 13, depicting a TIAS/TARP-BPSR chart. Step (1) user holds or connected to the device 106/107 on smartphone 100, in a step (2) the device 100 detects presence of the user, step 3) device 100 measures and system 10 recognizes the behavioral physiological parameters related to in TIAS or TARP stored protocols on application software 115C or in Claud 112F, 4) system triggers an “alert.” 5) The user can recognize the “alerts” (for example a voice, sound, light, vibration or image message alert) from the device or on the smartphone screen that automatically trigger the user to respond by executing the related self-regulation TIAS or TARP. Step (6) The system measure in real-time and compare 117 and provide rating in 112F with previous TIAS and TARP. 7) The system 10 and method 20 enables user to make personalized modifications in real-time in the biofeedback protocol as executing the TIAS or TARP to optimize success rating. 8) The system is configured to provide success rate and also reinforce good habits by displaying motivational reinforcements on the smartphone. User confirm with CR or NAR. 9) if NAR and user to continue, ask an AEFDQ and restart at step (3), as shown exemplarily in the flowchart of FIG. 14.

FIG. 15 shows an example of a contextual grid. Keeping focused on the behavioral goals of the users, the system requires to ensure measurable objectives and action steps build into coaching process setting goals, taking actions and tracking progress, and this can be facilitated by the use of a contextual grid. Learn about the contextual preference, a gap between current performance and desired performance, discover situations where the impact is difference from interaction, gain clarity about behaviors that users would like to change. What can you do about it? Can't do anything at all—but important—do it. Can't do anything at all—but not important—drop it. What deeper values do you find in the task? Self-awareness, put your experience in a longer context, make user understand, become a habit.

According to some aspects of the present invention, as explained above, a system 10 or a method 20 is provided, for actively monitoring a health of a user via a portable electronic device, for example a smartphone 100, to receive repeatedly context-based data from user that is related to a plurality of health parameters HP, to determine and provide for training instructions to the user to self-regulate his health and healthy behavior in real-time, for example by SRTP. The system 10 can be configured to receive multilevel data relating to physical, mental and emotional health status, activity level and wellbeing status of the user from inputs coming from different sensors 106, 107 as health parameters HP, or from questions that are prompted to the user, user profile indicators and via an interactive process with the user to determine specific contextual inputs important for real-time analyze, feedback, suggestion of self-regulation solutions and measure progress, to determine specific health segments HS.

The system 10 and method 20 is configured to process simultaneously multilevel data, and provide an individualized identification of needs based on the user profile, user baseline and historical data stored in application software of smartphone 100 or on cloud server 130, stored in cloud and displayed in application software, to determine highly personalized intervention via feedback, suggested instant action solutions (IAS) and suggested training protocols (STP) to overcome a unhealthy problem or behavior, and capable to train and track the achieved measured effects, during (pre) or after (post) intervention improving physical, mental or emotional health status of the user, and the selected IAS or STP, to be proposed again for an upcoming similar event/signs of needs and gradually transform them into trained instant action solutions (TIAS) and trained automated response patterns (TARP).

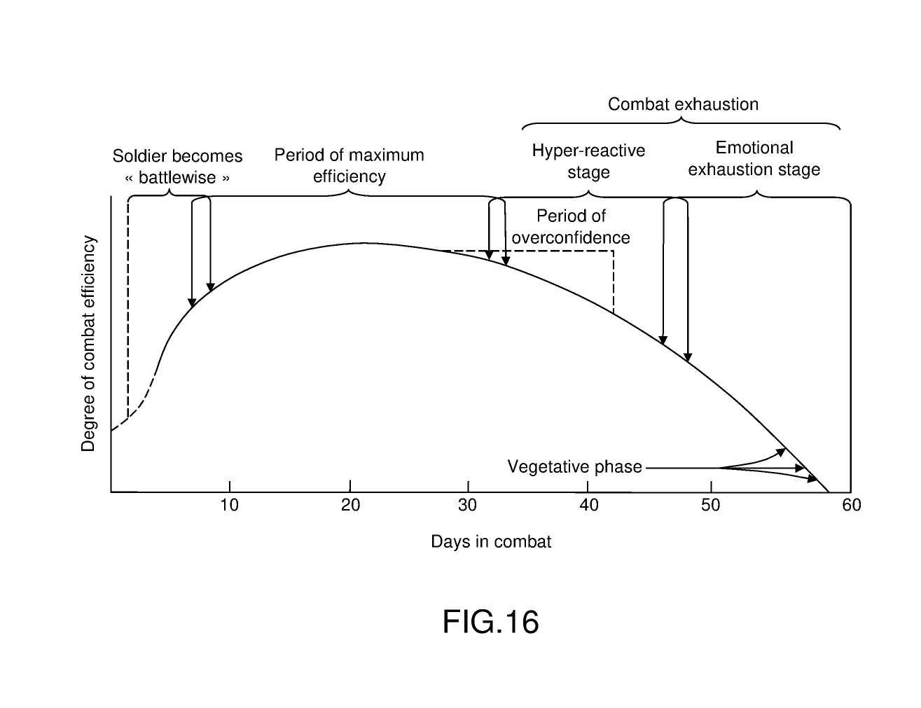

According to at least some aspects of the present invention, substantial advantages are provided for reducing the need of a trainer and therapist present during the session, and advantages for self-learning of the user, at anywhere and anytime by guiding the user to develop healthy living routines to foster a healthy lifestyle and overcome unhealthy patterns or behavior. The system 10 and method 20 and will take over that role to a certain level and thanks to its high level of accuracy in measuring and its comprehensive algorithm in processing data via multiple components in the system 10 and method 20, for example including a smartphone 100, measuring devices and sensors 101, application 102 of smartphone 103, and cloud server 103, by testing, teaching, train and track the user through an instant or full self-regulation training process. System 10 and method 20 is also able to give a global, holistic, integrated and/or three-hundred and system (360) approach of the overall health of user and wellbeing status based on ten (10) health segments HS that indicate human health risk and behaviors, and indicate the users daily living index and where the user is located on the human performance or human function curve, as shown in FIG. 16. For example, the user daily living index can be part of the second detailed feedback, or can be constantly displayed by the graphical user interface 111 in a dashboard, status bar, etc., and can indicate value that is indicative of the overall health of the user and their current health patterns. This value can be calculated based on the health segments SM, for example an average value, or a weighted average value of the health segments.

According to at least one embodiment, a more self-therapy process is provided helping the user to overcome various problem through a more in depth evaluation with contextual categories to bring out a highly individualized and personalized solutions brought forward thanks the algorithms of the method, represented by steps S72 to S76. When physical, mental and emotional health is monitored and occurs in different segments of a person health and behavioral parameters during different scenarios, stressors and factors integrated in the users daily life, system 10 and method 20 can detect early maladaptive behavioral physiological patterns through physiological measured signals such as but not limited to HR, HRV, ANS, and RSA, blood pressure wave or device, voice waves when user speaking on the smartphone, activity level or sedentary for a longer period, body weight and body composition, sleeping pattern, and level of self-care including smoking, drinking and illness through questionnaires, and make user aware to want to overcome detected maladaptive patterns, with a process that interacts with measuring devices 106, 107, smartphone 100, application software 102, protocols and cloud server algorithm and internet platforms, and thanks to the systems structure and interaction with user to make user aware of the unhealthy trigger points early to inappropriate response that can be related to an activity, for example but not limit public speaking, not master a sport, fear of animals, an event, for example but not limited to flying, dark room, go to a party, or a feeling, for example but not limited to pressure, not in control, fear of failing or an early health problem indicator like, for example extra systolic beats due to prolonged stress, lack of recuperation and increased body tension.

The system 10 and method 20 can also detect unhealthy patterns when the user is measures the health parameters HP and determines values for the ten (10) health segments during a “retreat” when system prompt user to follow a specific set frequencies to obtain a best condition to measure in-order obtain an optimal and most accurate baseline. Many studies have demonstrated extreme inaccuracy in many self-measuring and self-reporting apps exist. However, the present system 10 and method 20 can provide for a more comprehensive technological to analyze the data, based on four (4) different components in the system: (1) built in sensors 106 in smartphone or tablet 100, or external devices 107, (2) intelligent application software 102, (3) cloud server 130 and the algorithms associated thereto, and (4) interactivity with the user.

With the system 10 and method 20, it is possible to go from simple self-monitoring to self-mastery, by going beyond tracking of certain parameters to go to a support of behavioral changes. The behavioral physiology response patterns are an important indicator of a person's health, energy and wellbeing state. This has been overlooked in favor of just monitoring physical health by most of the existing self-monitoring solutions. The features described herein are classified into four categories: (1) instant action solutions (2) self-regulation training protocols (3) self-therapy journey (4) consultation by health professional.

With the system 10 and method 20, it is possible to measure different health parameters HP in real-time, for example heart-rate, heart rate variability, respiratory sinus arrhythmia, breathing rate, and sympathetic and parasympathetic nervous system activity of the autonomic nervous system (ANS). We can determine level of energy, stress and fatigue via the sympathetic branch considered as the “fight or flight” and the parasympathetic branch considered as the “rest and digest”, based on the knowledge graph of the health segments (HS). Autonomic functions include control of respiration, cardiac regulation, vasomotor (actions upon a blood vessel which alter its diameter) and certain reflex action such as coughing, swallowing and vomiting.

According to an aspect of the present invention, with the system 10 and method 20, when using contextual parameters like location, interaction with people, activity, availability, environmental and link to symptoms of physical, mental, emotional and behavioral responses visualized on graphical user interface 111 of smartphone 100 helped client to become more aware, supported by their understanding how various contextual aspects in their lives can influences their behavioral physiology response, health parameters and status of being—well (how they feel, think, behave and perform) and how their physiological condition interrelated with their mental capacity, emotional stability and behavioral actions (engagement) in how they responded to a stressor, to a challenge or resilience in ability to cope.

Using advanced wearable technologies linked to our intelligent interactive software solution consisting of a cloud and app with a built-in algorithm for realization of a self-monitoring toward self-mastery in a system that helps people become more in charge of their present state and ability to change maladaptive behavioral physiological response patterns to create solutions that deal with, early detection or prevention from various chronic health aspects. Engaging the user in a change process—become involved—contextual inputs to identify “stressors”, describe, develop and act in control. Holistic approach to analyze all present and history data—and highlight the specific need and also the underlying cause, to bring forward a better understanding toward a change rather than looking at each aspects (segment) in isolation. Support self-understanding, self-reflection and self-desire (willingness to create self-responsibility) by identify various negative stressors, triggers, behavior, responses and patterns within daily living activities.

Integrate biofeedback screens to offer a different dimension and forms for learning when using captured data into a displaying protocol in real-time, make new learned and to become automatically adaption to the parameters of the user and gradually guide the user to an optimal comfortable state alternating between “comfort-challenge-panic” to identify limits and personal optimal behavioral physiology response. Developing awareness and mapping out actions to create self-responsibility from identify needs, analyzed effects and developed goals.

The system learning (SL) of the steps S72, S74, and S76 enables the application to seamlessly get to know the user. After a first a set of introductory questions and answers, for example the self-care inventory questionnaire, away for the SL to learn to know the user, create an overall picture of the ten health segments HS and underlying parameters, the learning loop continues by start recording the different measures and comparing with SCI results, and then directly provides a raw feedback to the user to check its understanding of the results. Once the user confirms that the raw feedbacks are relevant, the SL asks for more contextual information about the measure, to learn a build the user profile. Based on the user profile, the population profile and the context, the learning loop interprets the measure results and gives feedforwards and some suggested actions.

Depending on the measure results and the history of measures, the SL will provide different advices: Instant action solution (IAS), to quickly improve the user state of mind, for example in a case of stress, self-training protocol (STP), allowing the user to learn about himself and practice small exercises every day, self-therapy journey (STJ), a suggested therapy program specifically designed to treat the user condition, in some weeks or months, Consultation, a warning and a suggestion to consult with a health professional when a medical condition is reported or known by the user or already followed by a doctor or when a normal value is considered as a medical concern by the systems algorithm and best practice is to consult with an specialized expert for more tests.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

1. An automated method for personal health management of a user having a personal user profile, the method performed on a computing device, the method comprising the steps of:
repeatedly measuring a plurality of health parameters by a mobile electronic device, the health parameters including a heart rate variability, a blood pressure, a motion activity, and a weight of the user;
calculating a base line dataset by the computing device for each one of the plurality of health parameters for a predetermined period of time;
comparing recently measured health parameters from the step of repeatedly measuring with the base line dataset;
providing on a display that is operatively connected to the computing device a first raw feedback on a health performance of the user based on the step of comparing;
repeatedly prompting the user to answer contextual questions, the contextual questions related to at least one of physical, mental, emotional, and behavioral status of the user; and
generating a second detailed feedback based on the personal user profile, the baseline data set, the measured health parameters, and a given context, to determine values for a plurality of health segments, and providing the second detailed feedback to the display of the user, based on a request by the user that includes the given context and data of the plurality of health segments.

2. The automated system according to claim 1, wherein the health segments include at least one of energy, stress, activity, weight, general health indicators, sleep, smoking, alcohol, self-care, and engagement.

3. The automated method according to claim 2, wherein one of the health parameters includes a mind-body coherence that is measured based on an activity of the autonomous nervous system of the user.

4. The automated method according to claim 2, wherein the second detailed feedback includes instructions to engage in a response pattern that a reduces a negative health impact.

5. The automated method according to claim 4, wherein the instructions to engage in the response pattern includes at least one of instructions to manage a breathing pattern, instructions to focus on a factor of creation of stress of the context, and instructions to engage in a visualization of an event of the context.

6. The automated method according to claim 1, wherein the contextual questions include questions regarding a transportation situation of the user and questions on an emotional mindset of the user.

7. The automated method according to claim 6, wherein the transportation situation includes use of a car, use of public transport, use of an airplane, walking or biking to a destination.

8. The automated method according to claim 1, wherein the personal user profiles includes an age, gender, and data on base line data set history and context.

9. The automated method according to claim 1, wherein the second detailed feedback is generated by subjecting history data of the plurality of health parameters to a decision tree, a knowledge graph for weighing and indicating relationships between the health parameters to link them to the health segments, and to a neural network that is trained to minimize a health impact based on the personal user profile.

10. An automated system for personal health management of a user having a personal user profile, the system comprising
a data processing device;
a user interface device in operative connection with the data processing device; and
a sensor device in operative connection to the data processing device, configured to measure a plurality of health parameters of the user,
wherein the sensor device is configured to repeatedly measure the plurality of health parameters, the health parameters including a heart rate variability, a blood pressure, a motion activity, and a weight of the user,
wherein the data processing device is configured to
calculate a base line dataset by the computing device for each one of the plurality of health parameters for a predetermined period of time;
compare recently measured health parameters from the step of repeatedly measuring with the base line dataset;
provide via the user interface device a first raw feedback on a health performance of the user based on the step of comparing;
repeatedly prompt the user to answer contextual questions via the user interface device, the contextual questions related to at least one of physical, mental, emotional, and behavioral status of the user; and
generate a second detailed feedback based on the personal user profile, the baseline data set, the measured health parameters, and a given context, to determine values for a plurality of health segments, and providing the second detailed feedback the user interface device, based on a request by the user that includes the given context and the health segments.

11. The automated system according to claim 10, wherein the health segments include at least one of energy, stress, activity, weight, general health indicators, sleep, smoking, alcohol, self-care, and engagement.

12. The automated system according to claim 11, wherein one of the health parameters includes a mind-body coherence that is measured based on an activity of the autonomous nervous system of the user.

13. The automated system according to claim 11, wherein the second detailed feedback includes instructions to engage in a response pattern that a reduces a negative health impact.

14. The automated system according to claim 13, wherein the instructions to engage in the response pattern includes at least one of instructions to manage a breathing pattern, instructions to focus on a factor of creation of stress of the context, and instructions to engage in a visualization of an event of the context.

15. The automated system according to claim 10, wherein the contextual questions include questions regarding a transportation situation of the user and questions on an emotional mindset of the user.

16. The automated system according to claim 15, wherein the transportation situation includes use of a car, use of public transport, use of an airplane, walking or biking to a destination.

17. The automated system according to claim 10, wherein the personal user profiles includes an age, gender, and data on base line data set history and context.

18. The automated system according to claim 10, wherein the second detailed feedback is generated by subjecting history data of the plurality of health parameters to a decision tree, a knowledge graph for weighing and indicating relationships between the health parameters, and to a neural network that is trained to minimize a health impact based on the personal user profile.

19. A non-transitory computer-readable medium having computer instruction code recorded thereon, the instruction code configured to perform the method of claim 1 when executed on a computer device.
